(19) United States
(12) Patent Application Publication
(10) Pub. No.: US 2020/0046720 A1
(43) Pub. Date: Feb. 13, 2020

(54) ENGINEERED BIOCOMPATIBLE ANTIBIOTIC PARTICLES AND THEIR USE AGAINST URINARY TRACT INFECTION

(71) Applicants: The University of North Carolina at Chapel Hill, Chapel Hill, NC (US); Duke University, Durham, NC (US)

(72) Inventors: Joseph DeSimone, Chapel Hill, NC (US); James Luft, Chapel Hill, NC (US); Ying Wang, Chapel Hill, NC (US); Patrick Seed, Chicago, IL (US)

(21) Appl. No.: 16/344,739

(22) PCT Filed: Oct. 25, 2017
(86) PCT No.: PCT/IB2017/056604

§ 371 Date: Apr. 24, 2019

Related U.S. Application Data
(60) Provisional application No. 62/412,535, filed on Oct. 25, 2016.

(51) Int. Cl. A61K 31/5383; A61K 9/14; A61K 47/14; A61K 47/34

(52) U.S. Cl. CPC A61K 31/5383; A61K 47/34; A61K 47/14; A61K 9/14

(57) ABSTRACT

Described herein, to overcome the limitations of conventional antibiotics, antibiotics have been encapsulated in biocompatible particles manufactured using Particle Replication In Non-wetting Templates (PRINT). These PRINT antibiotic particles were assessed as a topical agent to prevent E. coli infection using in vitro and in vivo models relevant to UTI and neurogenic bladder. The results show a prolonged efficacy and wide distribution in the bladder, resulting in a prophylactic environment in the bladder. The subject matter described herein is directed to molded particles containing an antibiotic active agent and methods of treating diseases and conditions with the particles, and methods of preparing the particles and compositions comprising the particles.

### GOVERNMENT SUPPORT

This invention was made with government support under Grant No. W81XWH-31-1-0450 awarded by the U.S. Army Medical Research and Materiel Command. The government has certain rights in the invention.

### FIELD OF THE INVENTION

The subject matter described herein is directed to molded particles containing an antibiotic active agent and methods of treating diseases and conditions with the particles, and methods of preparing the particles and compositions comprising the particles.

### BACKGROUND

Urinary tract infection (UTI) is among the most common infectious disease in humans, costing over $3 billion annually in the US [1]. In otherwise healthy females, it is a highly recurrent disease approximately 20-30% of individuals having serial infections and 5% having chronic recurrent disease [2,3]. Among individuals with neurogenic bladder, in which immune dysfunction and incomplete micturition greatly increase susceptibility to UTI, recurrent disease is a life-long problem that may precede urosepsis and renal failure [4]. Sustained oral prophylactic antibiotics are often used as one of only a few options to prevent recurrent UTI. Oral antibiotics have a major impact on the intestinal microbiota and thus have limited utility due to cumulative emergence of antibiotic-resistant organisms and intolerable side effects [5,6]. Furthermore, cumulative evidence indicates that uropathogenic organisms such as Escherichia coli and Klebsiella pneumoniae invade into the uroepithelium lining in the urinary tract where they subvert innate immune clearance and persist despite attempts at treatment with many clinical antibiotics, which are relatively ineffective against intracellular bacteria [7-10].

Topical or intracystic therapies have been used to prevent recurrent UTI in the neurogenic bladder and have included instillation of antibiotics or intentional colonization with competitive non-pathogenic bacteria (bacterial interference) to prevent colonization with uropathogenic organisms [11,12]. Topical antibiotic therapy has the benefit of avoiding exposure of the oral, intestinal, and vaginal microbiota to the antibiotic, thus limiting the emergence of resistant organisms from these reservoirs. Topical therapy also avoids potential side effects and drug interactions that may occur during systemic therapy including oral and intravenous routes of administration.

Past uses of topical therapy for UTI prophylaxis has however had several limitations. Aminoglycosides have commonly been instilled into the bladder for UTI prevention; however, this class of antibiotics has microbiologically insignificant intracellular activity, which is now well appreciated as a bacterial niche during UTI [7,9]. Furthermore, the effectiveness of aminoglycosides and other intracystic antibiotics such as neomycin and polymyxin B is limited by the dwell time, which is typically only a short period each day (often during sleep) or every several days. The accumulation of urine in the bladder dilutes the intracystic antibiotic and bladder emptying, by catheterization or spontaneous voiding, results in a washout of the antibiotic, thus leading to troughs in the minimum concentration of antibiotic needed to prevent infection and increasing the window of susceptibility to UTI [13-15]. Bacterial interference has been limited by inconsistent colonization of the bladder by the competitive non-pathogenic organism and by the potential risks for evolution of the therapeutic bacteria strain [16,17].

The antibiotic levofloxacin has multiple desirable features as related to the prevention and treatment of UTI, including a spectrum of activity inclusive of most of the predominant uropathogens in community-acquired, hospital acquired, and special population (like nursing home and SCI/neurogenic bladder) UTI. Levofloxacin has good intracellular and extracellular activity. Yet, like many other antibiotics, resistance can emerge after repeated oral exposures [18]. Also, levofloxacin has significant drug-drug interactions that are important in medically complex patients (often accompanied by neurogenic bladder) and can promote Clostridium difficile infection [19].

What is therefore needed is a means for establishing and maintaining an effective amount of antibiotic in the bladder to provide a therapeutic and even prophylactic effect. The subject matter described herein addresses this shortcoming in the art.

### BRIEF SUMMARY

In embodiments, the subject matter described herein is directed to engineered particles comprising an effective amount of an antibiotic.

In embodiments, the subject matter described herein is directed to methods of treating a UTI in a subject in need thereof by administering a composition comprising engineered particles comprising an effective amount of an antibiotic.

In embodiments, the subject matter described herein is directed to a method of preventing a recurrence of a UTI by administering a composition comprising engineered particles comprising an effective amount of an antibiotic, wherein an effective level of antibiotic is maintained in the desired tissue/organ for up to 24 hours post administration.

In embodiments, the subject matter described herein is directed to methods of preparing engineered particles comprising an effective amount of an antibiotic.

Other embodiments are also described.

### BRIEF DESCRIPTION OF THE FIGURES

FIGS. 1A-C show scanning electron microscopy image of the Levofloxacin 593 loaded PLGA-PRINT particles (FIG. 1A) 1×1 μm, (FIG. 1B) 2×0.6 μm and (FIG. 1C) 3×1 μm. The scale bars in each image represent 5 μm.

FIGS. 2A-C show growth of E. coli UT189 in media containing free or PRINT-598 Levofloxacin. FIG. 2A: CFU after 2 hr growth in liquid broth with free or PRINT encapsulated levofloxacin. Growth was measured by plating on solid agar media without antibiotics. Drug concentration denotes the final concentration of levofloxacin. FIG. 2B: Zone of inhibition for free (LEVO) or PRINT encapsulated levofloxacin formulations D1-7. Each disk contains 0.05 μg of levofloxacin. See main text for mean vales of zones of inhibition for each condition. *0.01<P<0.05; **0.01<P<0.001; ***P<0.001. FIG. 2C: A representative plate showing the zones of inhibition from disk disc diffusion of levofloxacin or a PRINT-Levofloxacin formulation.

FIGS. 3A-D show PRINT-Levofloxacin provides prolonged protection against intracellular E. coli infection. UROTSA bladder epithelial cell monolayers were incubated with media, free levofloxacin (FREE LEVO), blank PRINT particles (BLANK PRINT), or a formulation of PRINT encapsulated levofloxacin (PRINT-LEVO) for 1 hour. Each treatment was removed, the cells were washed, and the cells were immediately infected with E. coli UT189 (FIG. 3A), 8 hours later (FIG. 3B), or 18 hours later (FIG. 3C). After 1 hour of infection with E. coli, the medium was replaced with medium containing gentamicin to eliminate extracellular bacteria. Viable bacteria in the bladder epithelial cells were enumerated 24 hours later. The formulations and associated sizes and charges are shown for reference (FIG. 3D).

FIG. 4 shows PRINT-encapsulated levofloxacin has minimal toxicity to bladder epithelial cells. Bladder epithelial cell line UROTSA was incubated 4, 8, 18, or 24 hours with media, free levofloxacin (5 ug/ml), BLANK PRINT particles (equivalent wt/volume) as formula D5, a PRINT LEVO formulation (D1-7), or media containing 1% Triton-X 100 as a positive lysis control. Bars and error bars indicate the means and standard deviations of 3 independent trials.

FIGS. 5A-E shows localization of PRINT-Levofloxacin with E. coli in bladder epithelial cells. FIG. 5A: Bladder epithelial cell line 5637 was co-incubated with PRINT-LEVO formulation D8 containing Qdots for visualization (red) and GFP-expressing E. coli UT189 (green). Prior to imaging the cell nuclei were co-stained with DAPI. The main panel shows a 200× magnification. Inset panels are 400× magnifications. FIGS. 5B-E: Flow cytometry was performed with 5637 cells alone (FIG. 5B), with PRINT-LEVO (formulation D5; FIG. 5C), with GFP-expressing E. coli (FIG. 5D), or with PRINT-LEVO and GFP-expressing E. coli (FIG. 5E). Numbers indicate the percentage of cells in each quadrant.

FIGS. 6A-C show PRINT-LEVO provides sustained protection against E. coli UTI in the SCI rat. Female SCI rats were treated by transurethral catheterization and instillation of a volume equivalent to their previously determined residual urine volume containing PBS, PBS with free levofloxacin (5 μg/ml), PRINT-LEVO (formula D5), or BLANK PRINT particles (wt/vol equivalent to PRINT-LEVO). After 1 hour of treatment, all animals had their bladder creded to remove each treatment. The animals were infected with E. coli at increasing time intervals following the administration and drainage of the test or control agents from the bladder: 4 hours (FIG. 6A), 8 hours (FIG. 6B), or 18 hours (FIG. 6C). Bacteria were counted in the bladders and kidneys 24 hours after infection with E. coli by tissue homogenization and plating of dilutions on solid agar medium. Horizontal bars indicate the geometric means.

### DETAILED DESCRIPTION

The presently disclosed subject matter will now be described more fully hereinafter. However, many modifications and other embodiments of the presently disclosed subject matter set forth herein will come to mind to one skilled in the art to which the presently disclosed subject matter pertains having the benefit of the teachings presented in the foregoing descriptions. Therefore, it is to be understood that the presently disclosed subject matter is not to be limited to the specific embodiments disclosed and that modifications and other embodiments are intended to be included within the scope of the appended claims. In other words, the subject matter described herein covers all alternatives, modifications, and equivalents. In the event that one or more of the incorporated literature, patents, and similar materials differs from or contradicts this application, including but not limited to defined terms, term usage, described techniques, or the like, this application controls. Unless otherwise defined, all technical and scientific terms used herein have the same meaning as commonly understood by one of ordinary skill in this field. All publications, patent applications, patents, and other references mentioned herein are incorporated by reference in their entirety.

Neurogenic bladder is a major risk factor for recurrent UTI, leading to the clinical challenge of balancing UTI prevention and the emergence of antibiotic resistance due to a high exposure to antibiotics. As used herein, neurogenic bladder is a general term for abnormal urination caused by disorder of sympathetic nerve, parasympathetic nerve, etc. controlling the action of bladder and urethra.

Conventional antibiotic therapy to prevent recurrent UTI in patients with neurogenic bladder is complicated by the disruption of the normal microbiota and the emergence of drug resistance. Furthermore, conventional antibiotics, oral or intracystic typically given once per day for prophylaxis, provide limited exposure in the urinary tract due to either the inherent pharmacokinetics of the UTI agents or, in the case of intracystitic administration, the washout of the agents with bladder emptying.

Described herein are engineered and manufactured PRINT particles encapsulating the UTI antibiotic levofloxacin: 1) to allow the drug to be distributed as a topical application to the urinary epithelium where infection occurs; 2) to avoid disruption of the normal microbiota; 3) to mimic bacteria by being taken up into the bladder epithelium; and 4) to produce a depot effect where sustained release of the encapsulated antibiotic provides consistent protection from infection using infrequent dosing with a target of once per day protection from infection. The preclinical data demonstrate that this strategy is effective, broadly meeting these objectives, and is tolerated well without toxicity.

Despite in vitro liquid culture and agar disc diffusion experiments showing that free levofloxacin was slightly more effective at killing E. coli than equivalent amounts of PRINT-Levofloxacin formulations, we found that PRINT-Levofloxacin was significantly superior to the unencapsulated free drug in vivo in a bladder epithelium tissue culture model.

In contrast, free levofloxacin, which likely achieves good intracellular concentrations like other fluoroquinolones, is reduced rapidly to ineffective concentrations when the environment external to the bladder epithelial cells is exchanged for drug-free media, similar to the wash out effect that occurs in the urinary tract as new urine is made and voided, creating an effective volume exchange. The experiments replicate these observations in the SCI rat, showing that PRINT-Levofloxacin provides extended protection against E. coli UTI when the effectiveness of unencapsulated levofloxacin is long past.

In summary, the data provided herein show that PRINT® (Particle Replication In Non wetting Templates) technology [20-22] can be used to fabricate antibiotic, e.g., levofloxacin, loaded poly(D,L-lactide-co-glycolide)s (PLGA) particles for topical, intracystic prophylaxis against UTI in medically complex patients (e.g. those suffering from spinal cord injury with neurogenic bladder). Encapsulation of levofloxacin in PRINT particles would facilitate the association and uptake of the particles into bladder epithelial cells where uropathogens invade to avoid typical immune responses and traditional therapies. Furthermore, PRINT encapsulation would provide a depot-effect whereby a single administration of the encapsulated antibiotic would provide prolonged protection. Herein, we developed levofloxacin PRINT particles in multiple formulations and dimension and assessed the effectiveness of the formulations in preventing infection by uropathogenic E. coli in vitro and in vivo in a model of UTI in rats with spinal cord injury. Compared to unencapsulated, soluble levofloxacin, PRINT-Levofloxacin provided lower inhibition of E. coli in liquid culture or disk diffusion assays. However, PRINT-Levofloxacin provided a durable depot effect in cultured bladder epithelial cells, protecting against E. coli intracellular infection for up to 18 hours after administration. Under similar conditions, unencapsulated levofloxacin lost significant effectiveness within 1 hour of a single dose. Further analysis confirmed that PRINT-levofloxacin had no cytotoxicity to cultured bladder epithelial cells at concentrations that were protective against bacterial infection.

A combination of microscopy and flow cytometry demonstrated that a single dose of PRINT-Levofloxacin was widely distributed throughout bladder epithelial cells and that the particles did not need to be co-localized with E. coli within infected cells to exert a protective effect. In a model of UTI in the spinal cord injured rat with neurogenic bladder, a single intracystic dose of PRINT-Levofloxacin provided 100% and 58% protection against E. coli infection at 8 hours and 18 hours, respectively. In contrast, a single intracystic dose of unencapsulated levofloxacin provided no prophylaxis even 4 hours post-administration. PRINT Levofloxacin shows efficacy in preclinical studies as an effective topical prophylactic agent to prevent E. coli urinary tract infection in the host with neurogenic bladder.

In embodiments, the subject matter described herein is directed to engineered particles comprising an effective amount of an antibiotic. In embodiments, the antibiotic is an antibiotic as described elsewhere herein. In embodiments, the antibiotic is levofloxacin.

In embodiments, the subject matter described herein is directed to methods of treating a UTI in a subject in need thereof by administering a composition comprising engineered particles comprising an effective amount of an antibiotic. In embodiments, the subject has suffered a spinal cord injury. As used herein, the term “spinal cord injury” refers to a condition occurring when a traumatic event damages cells within the spinal cord, or when the nerve tracts that relay signals up and down the spinal cord are severed or otherwise injured. Some of the most common types of spinal cord injury include contusion and compression. Other types of injuries include, but are not limited to lacerations, and central cord syndrome.

In embodiments, the subject matter described herein is directed to a method of preventing a recurrence of a UTI by administering a composition comprising engineered particles comprising an effective amount of an antibiotic. In this aspect, the method comprises establishing and maintaining an effective amount of antibiotic in the bladder for up to 24 hours. In certain aspects, the effective amount of antibiotic in the bladder is maintained for up to 20, 18, 16, 14, 12, 10 8, 6, 4, 2 or 1 hour(s).

In embodiments, the subject matter described herein is directed to methods of preparing engineered particles comprising an effective amount of an antibiotic.

As used herein, the term “particle” or “particles” is intended to mean one or more molded particles. The particles can comprise a polymer matrix. The methods and materials for fabricating the particles of the present invention are further described and disclosed in patent applications, each of which are incorporated herein by reference in their entirety: U.S. Pat. Nos. 9,340,001; 9,214,590; 9,205,594; 8,992,992; 8,945,441; 8,662,878; 8,518,316; 8,444,907; 8,444,899; 8,439,666; 8,420,124; 8,268,446; 8,263,129; 8,158,728; 8,128,393; 7,976,759; U.S. Pat. Application Publications Nos. 2016-0236379, 2016-0059473, 2015-0283079, 2014-0027948, 2013-0209564, 2013-0228950, 2013-0011618, 2011-151015, 2010-0003291, 2009-0165320; and PCT Publication No. WO2015/073831.

Particles described herein comprise an active agent dispersed in a biocompatible matrix. The biocompatible matrix is formed from one or more materials that are compatible with living tissue or a living system and are preferably not toxic, injurious or physically adversely reactive. Particles of the invention are also compatible with living tissue or a living system. However, some degree of toxicity, injury, or physical adversity may be tolerated, such as that necessary to diagnose, cure, mitigate, treat, or prevent a disease in an organism.

In some embodiments, the biocompatible matrix is comprised of one or more polymers. The polymer may be a homopolymer, or a hetero- or co-polymer, such as an alternating or block copolymer. Preferably, such polymeric matrix is biocompatible, biodegradable, bioresorbable, biodissolvable, and/or bioclearable in or from the human body. Suitable biocompatible polymers include polymers selected from the group consisting of a polyester, a polyanhydride, a polyamide, a phosphorous-based polymer, a poly(cyanoacrylate), a polyorthoester, a polyurethane, a polyorthoester, a polyether, a carbohydrate, a polypeptide, a hydroxypropylcellulose, a poly(ethylene glycol), a wax, a hydrogel, a phosphatidylcholine, a polydihydropyran, a polyacetal, a biodegradable polymer, and combinations thereof. In some embodiments, the polyester is selected from the group consisting of polylactic acid, polylactide, polyglycolic acid, poly(hydroxybutyrate), poly(ε-caprolactone), poly(β-L-malic acid), polydioxanone, poly(lactide-co-glycolide) polymer, and polyhydroxyalkanoate. In some embodiments, the polyanhydride is selected from the group consisting of poly(sebacic acid), poly(adipic acid), and poly(terephthalic acid). In some embodiments, the polyamide is selected from the group consisting of poly(imino carbonates) and polyaminoacids. In some embodiments, the phosphorous-based polymer is selected from the group consisting of polyphosphate, a polyphosphonate, and a polyphosphazene. In embodiments, the polymer is a polyester. In preferred embodiments, the polymer is a poly(lactide-co-glycolide) polymer.

In embodiments, the biocompatible matrix further comprises cationic agents. Cationic agents includes cationic lipids, cationic polymers, cationic lipidoids, and cationic agents containing a portion having a positive charge in aqueous solutions at neutral pH.

Suitable cationic lipids includes, but is not limited to N-[1-(2,3-dioleyloxy)propyl]-N,N,N-trimethylammonium chloride (DOTMA); dioleyldimethylammonium chloride (DODAC); N-oleyl,N-stearyl-N,N-dimethylammonium chloride (OSDAC); distearyldimethylammonium chloride (DSDAC); 3-[N—(N′,N′-dimethylaminoethane)-carbamoyl] cholesterol hydrochloride (DC-Chol); dioleoylphosphatidylserine (DOPS); dioleoylphosphatidic acid (DOPA); 1-palmitoyl-2-oleoylphosphatidylglycerol (POPG); phosphatidylinositol (PI); tetraoleoylcardiolipin (CL); sn-(3-oleoyl-2-hydroxy)-glycerol-1-phospho-sn-3′-(1′-oleoyl-2′-hydroxy)-glycerol (lysobisphosphatidic acid; S,R isomer); dioleoylphosphatidylcholine (DOPC); dioleoylphosphatidylethanolamine (DOPE); dipalmitoylphosphatidylserine (DPPS); dimethyldioctadecylammonium (DDAB); cholesterol; I,2-dioleoyl-sn-glycero-3-ethylphosphocholine (EPC); I,2-di-(9Z-octadecenoyl)-3-dimethylammonium-propane (DODAP); I,2-dilinoleyloxy-3-dimethylaminopropane (DLinDMA) and derivatives thereof; 1,2-dioleoyl-3-trimethylammonium-propane (chloride salt) (DOTAP); and 1,2-dioctadecanoyl-sw-glycero-3-phosphocholine (DSPC); 1,2-distearoyl-sn-glycero-3-phosphoethanolamine (DSPE). In embodiments, the cationic lipid is cholesterol, 3-[N—(N′,N′-dimethylaminoethane)-carbamoyl] cholesterol hydrochloride (DC-Chol), or 1,2-dioleoyl-3-trimethylammonium-propane (chloride salt) (DOTAP). In preferred embodiments, the cationic lipid is 1,2-dioleoyl-3-trimethylammonium-propane (chloride salt) (DOTAP).

Suitable cationic polymers include, but are not limited to polycation-containing cyclodextrin, histones, protamines, cationized human serum albumin, aminopolysaccharides such as chitosan, peptides such as poly-L-lysine, poly-L-ornithine, and poly(4-hydroxy-L-proline ester, and polyamines such as polyethylenimine (PEI; available from Sigma Aldrich), polypropylenimine, polyamidoamine dendrimers (PAMAM; available from Sigma Aldrich), cationic polyoxazoline and poly(beta-aminoesters).

Suitable cationic lipidoids include, but are not limited to cationic lipidoids (as described by K. T. Love in the publication PNAS 107, 1864-1869 (2010)). Other exemplary cationic polymers include, but are not limited to, block copolymers such as PEG-PEI and PLGA-PEI copolymers.

Suitable cationic agents containing a portion having a positive charge in aqueous solutions at neutral pH include the following Compounds (A-I):

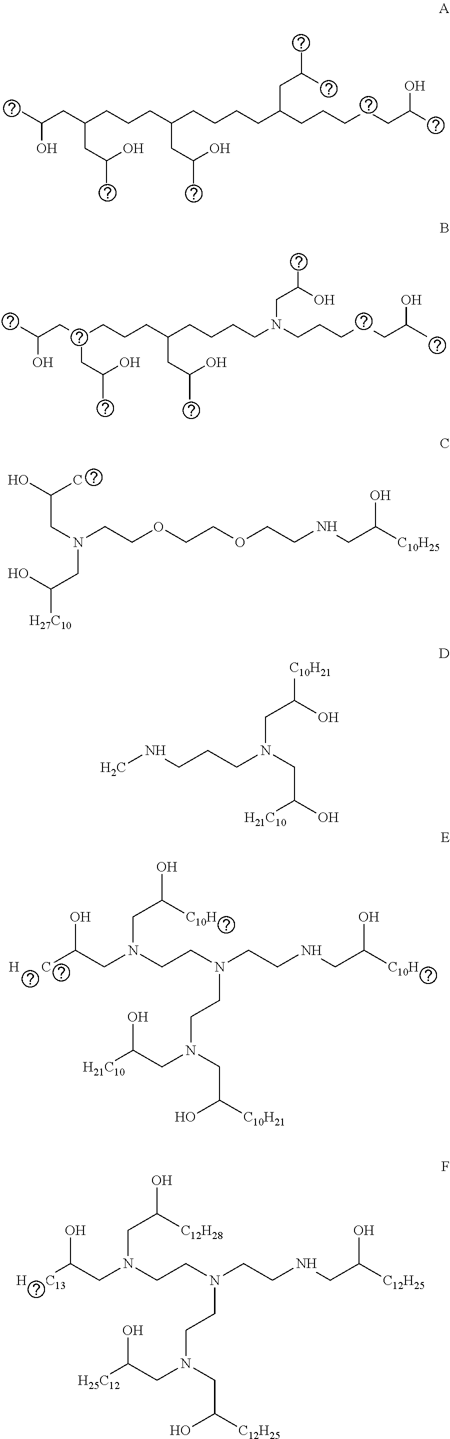

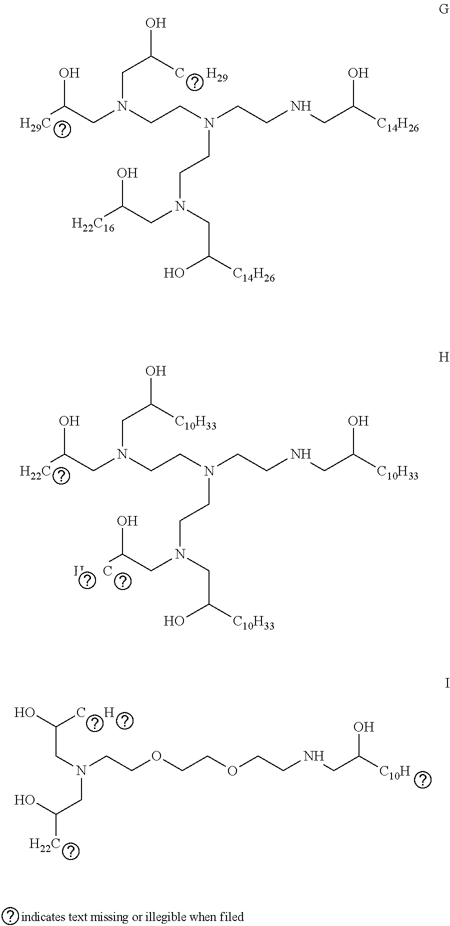

Additionally, other cationic agents include structures of the general Formula:

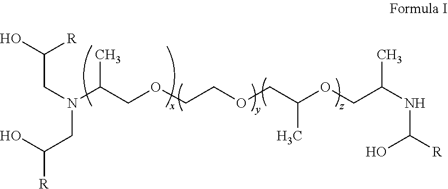

TABLE 1
Values for Variables x + z, y and R for Compounds J-R of Formula I.

| Compound | x + z | y | R |
|---|---|---|---|
| Compound J | 6 | 12.5 | C₁₂H₂₅ |
| Compound K | 1.2 | 2 | C₁₂H₂₅ |
| Compound L | 6 | 39 | C₁₂H₂₅ |
| Compound M | 6 | 12.5 | C₁₄H₂₉ |
| Compound N | 1.2 | 2 | C₁₄H₂₉ |
| Compound O | 6 | 39 | C₁₄H₂ |
| Compound P | 6 | 12.5 | C₁₆H₃₃ |
| Compound Q | 1.2 | 2 | C₁₆H₃₃ |
| Compound R | 6 | 39 | C₁₆H₃₃ |

Cationic agents, such as those listed above, can generally be prepared by the reaction of an appropriate hydrophobic epoxide (e.g. oleyl epoxide) with a multifunctional amine (e.g. propylene diamine). Details of the synthesis of related cationic agents are described by K. T. Love in the publication PNAS 107, 1864-1869 (2010) and Ghonaim et al., Pharma Res 27, 17-29 (2010).

It will be appreciated that polyamide derivatives of PEI (PEI-amides) can also be applied as cationic agents. PEI-amides can generally be prepared by reacting PEI with an acid or acid derivative such as an acid chloride or an ester to form various PEI-amides. For example, PEI can be reacted with methyl oleate to form PEI-amides.

In addition to a biocompatible matrix, particles of the invention may comprise an active agent. Active agents may be agents used to diagnose, cure, mitigate, treat, or prevent a disease in an organism. Active agents may be diagnostic agents, therapeutic agents, and/or drugs. In embodiments, the active agent is a drug. In preferred embodiments the active agent is an antibiotic.

Antibiotic referred to herein is a substance derived naturally from fungi or bacteria, or synthetically, that destroys or inhibits the growth of microorganisms. General classes of antibiotic include, but are not limited to, β-lactam antibiotics, polypeptides and quinolones. More specifically, the antibiotic may be selected from the group consisting of penicillins, cephalosporins, carbepenems, beta-lactams antibiotics, aminoglycosides, amphenicols, ansamycins, macrolides, lincosamides, glycopeptides, polypeptides, tetracylines, chloramphenicol, quinolones, fucidins, sulfonamides, sulfones, nitrofurans, diaminopyrimidines, trimethoprims, rifamycins, oxalines, streptogramins, lipopeptides, ketolides, polyenes, azoles, echinocandins, and any combination thereof. Antibiotics, in particular, β-lactam antibiotics, polypeptides, quinolones, or mixtures thereof, are especially potent against a broad spectrum of microorganisms, i.e., effective against a variety of microorganisms, particularly, against both gram-negative and gram-positive bacteria.

As used herein, the antibiotic can be selected from the group consisting of penicillins, cephalosporins, carbepenems, other beta-lactam antibiotics, aminoglycosides, amphenicols, ansamycins, macrolides, e.g., Erythromycin, Clarithromycin and Azithromycin, lincosamides, glycopeptides, polypeptides, tetracylines, chloramphenicol, quinolones, fucidins, sulfonamides, sulfones, nitrofurans, diaminopyrimidines, trimethoprims, rifamycins, oxalines, streptogramins, lipopeptides, ketolides, polyenes, azoles, echinocandins, and any combination thereof. In embodiments, the antibiotic is selected from the group consisting of β-lactam antibiotics, polypeptides, quinolones, and any combination thereof.

Suitable antibiotics may be selected from the group consisting of azithromycin, ciprofloxacin, doxycycline hyclate, amoxicillin, cephalexin, levofloxacin, minocycline, nitrofurantoin, doxycycline, cefuroxime axetil, amoxicillin-potassium clavulanate, ciprofloxacin (mixture), erythromycin, nitrofurantoin monohydrate/macrocrystals, tetracycline, doxycycline monohydrate, trimethoprim, methenamine hippurate, cefadroxil, cefepime, ampicillin, sulfamethoxazole-trimethoprim, cefaclor, cefpodoxime, gentamicin, ceftriaxone, ciprofloxacin HCl, cefazolin, ofloxacin, erythromycin ethylsuccinate, eftriaxone, methenamine mandelate, meropenem, cefixime, ciprofloxacin, amikacin, erythromycin stearate, levofloxacin, cefepim, doxycycline hyclate, demeclocycline, aztreonam, tobramycin, ceftazidime, streptomycin, gentamicin sulfate, fosfomycin tromethamine, cefoxitin, erythromycin lactobionate, ampicillin, imipenem-cilastatin, tobramycin, cefepime injection, cefuroxime, cefazolin, ciprofloxacin, aztreonam, minocycline, cefuroxime sodium, ertapenem, gentamicin, ceftazidime, cefazolin, ceftriaxone, ertapenem, doripenem, cefazolin, doxycycline-benzoyl peroxide, and cefotaxime. In particular embodiments, the antibiotic is levofloxacin.

In some embodiments, the particles are highly uniform with respect to shape, size and/or composition. One way in which such particles may be fabricated is using PRINT™ Technology (Liquidia Technologies, Inc.), which is a method capable of forming particles that: (i) are monodisperse in size and uniform shape, (ii) can be molded into any shape, (iii) can be comprised of essentially any matrix material, in particular biocompatible materials, (iv) can be formed under mild conditions (compatible with delicate cargoes), (v) are amenable to post-functionalization chemistry (e.g. bioconjugation of antigens and/or targeting components), and (vi) which initially fabricates particles in an addressable 2D array.

As referred to herein, an amount, value or shape that is the “same,” “substantially the same” or “substantially similar” is one that does not vary in a significant way from a given reference point or value. With regard to particles formed by the present methods, the shapes and dimensions of the particles are reproducible and a plurality of particles is substantially the same in shape, size, and composition. A plurality of particles means at least two particles. Scanning electron micrography can be used to evidence the substantially similar nature of the particles even at nanometer resolution.

As used herein, the term “substantially mimicking” means a molded particle that has a shape that is predetermined from the mold used to prepare the particle. This term includes variance in the shape, size, volume, etc. of the particle from the mold itself. However, the particles shape, size, volume etc. cannot be random since they are prepared from molds and substantially mimic the mold's shape, size, volume, etc. The term “amorphous” refers to a shape that is not engineered. A shape that is not prepared from a mold can be amorphous. Amorphous shapes by definition cannot be systematically reproducible. This is in contrast to molded shapes.

According to some embodiments, the composition can further include a plurality of particles, where the particles have a substantially uniform mass, are substantially monodisperse, are substantially monodisperse in size or shape, or are substantially monodisperse in surface area. In some embodiments, the plurality of particles have a normalized size distribution of between about 0.80 and about 1.20, between about 0.90 and about 1.10, between about 0.95 and about 1.05, between about 0.99 and about 1.01, between about 0.999 and about 1.001. According to some embodiments, the normalized size distribution is selected from the group of a linear size, a volume, a three dimensional shape, surface area, mass, and shape. In yet other embodiments, the plurality of particles includes particles that are monodisperse in surface area, volume, mass, three-dimensional shape, or a broadest linear dimension.

Particle characteristics used to describe the shapes examined include: a) the shape diameter (SD); it is the minimum diameter of a circumscribed circle around the particle; b) the minimum feature size (MFS); it is the diameter of the smallest distinct geometry of the shape; and c) the volume of the shape. All of these characteristics can be readily determined by one of skill in the art using the information disclosed herein and information known in the art. In embodiments where the shape of the particle is essentially a rod, the particles can have aspect ratios calculated by the width×height. Aspect ratio refers to the ratio of the longest axis to the shortest axis of a particle. Aspect ratios for rod shapes will be >1:1. In embodiments, the aspect ratio is 2:1; 3:1; 4:1; 5:1; 6:1; 7:1, 8:1; 9:1; 10:1 and so on.

In some embodiments, the physical properties of the particle are varied to enhance cellular uptake. In some embodiments, the size (e.g., mass, volume, length or other geometric dimension) of the particle is varied to enhance cellular uptake. In some embodiments, the charge of the particle is varied to enhance cellular uptake. In some embodiments, the charge of the particle ligand is varied to enhance cellular uptake. In some embodiments, the shape of the particle is varied to enhance cellular uptake. In some embodiments, the physical properties of the particle are varied to enhance biodistribution. In some embodiments, the size (e.g., mass, volume, length or other geometric dimension) of the particle is varied to enhance biodistribution. In some embodiments, the charge of the particle matrix is varied to enhance biodistribution. In some embodiments, the charge of the particle ligand is varied to enhance biodistribution. In some embodiments, the shape of the particle is varied to enhance biodistribution. In some embodiments, the aspect ratio of the particles is varied to enhance biodistribution. In some embodiments, the physical properties of the particle are varied to enhance cellular adhesion. In some embodiments, the size (e.g., mass, volume, length or other geometric dimension) of the particle is varied to enhance cellular adhesion. In some embodiments, the charge of the particle matrix is varied to enhance cellular adhesion. In some embodiments, the charge of the particle ligand is varied to enhance cellular adhesion. In some embodiments, the shape of the particle is varied to enhance cellular adhesion.

The term “treating” as used herein refer to reduction in severity and/or frequency of symptoms, elimination of symptoms and/or underlying cause, prevention of the occurrence of symptoms and/or their underlying cause, and improvement or remediation of damage. Thus, for example, “treating” a patient involves prevention of a particular disorder or adverse physiological event in a susceptible individual as well as treatment of a clinically symptomatic individual by inhibiting or causing regression of a disorder or disease. As used herein the terms “treating” includes “ameliorating,” which refers to all processes wherein there may be a slowing, interrupting, arresting, or stopping of the progression of the condition or symptoms and does not necessarily indicate a total elimination of the underlying condition. In embodiments, the term “ameliorating” and “dampening” refer to a lessening of the severity of a symptom and there are clinical assessments and markers that can be used to identify and quantify the lessening of symptoms. Also included in the amelioration of symptoms is the perception by the subject that the symptoms have lessened.

The term “therapeutically effective amount” as used herein refers to an amount of the particles containing an antibiotic that is sufficient to achieve a certain outcome, such as to treat or prevent a UTI. The effective amount and dosage of such antibiotic(s) required to be administered for effective treatment are known in the art or can be readily determined by those of skill in this field. Of course, the amount of antibiotic administered will depend upon a variety of factors, including, for example, the particular indication being treated, the mode of administration, whether the desired benefit is prophylactic or therapeutic, the severity of the indication being treated and the age and weight of the patient, the bioavailability of the particular active compound, and the like. Determination of an effective dosage is well within the capabilities of those skilled in the art coupled with the general and specific examples disclosed herein. Where antibiotics do not have a known dosage for certain diseases, the effective amount of antibiotic and the amount of a particular dosage form required to be administered for effective treatment can be readily determined by those of skill in this field. Thus, the term “therapeutically effective amount” can mean an amount of a particles or antibiotic(s) within the particles that (i) treats the particular disease, condition, or disorder, (ii) attenuates, ameliorates, or eliminates one or more symptoms of the particular disease, condition, or disorder, or (iii) prevents or delays the onset of one or more symptoms of the particular disease, condition, or disorder described herein. A “therapeutically effective amount” of a particles or active agent(s) within the particles also means a nontoxic but sufficient amount of the agent to provide the desired effect.

For the prevention or treatment of a UTI, the appropriate dosage will depend on the patient, the severity and course of the disease, whether particles are administered for preventive or therapeutic purposes, previous therapy, the patient's clinical history, and the discretion of the attending physician. The particles are suitably administered to the patient at one time or over a series of treatments. Depending on the type and severity of the disease, about 1 μg/kg to 50 mg/kg (e.g. 0.1-20 mg/kg) of particles is an initial candidate dosage for administration to the subject, whether, for example, by one or more separate administrations, or by continuous infusion. In some embodiments, the dosage of the particles will be in the range from about 0.05 mg/kg to about 10 mg/kg. Thus, one or more doses of about 0.5 mg/kg, 2.0 mg/kg, 4.0 mg/kg or 10 mg/kg (or any combination thereof) may be administered to the subject. Such doses may be administered intermittently, e.g. every week or every three weeks.

The amount of antibiotic present in the pharmaceutical composition will depend on the antibiotic. Most useful antibiotics are indicated for certain diseases and conditions and the dose amount of active agent can be readily determined and a pharmaceutical composition comprising the desired amount can be prepared as disclosed herein. Useful values of antibiotics are from about 1 mg to about 1,500 mg antibiotic per dosage form of the pharmaceutical composition. Preferred values are from about 10 mg to about 800 mg.

The particles can be formulated into pharmaceutical compositions as described herein. In an embodiment, the subject matter disclosed herein is directed to a method of treating a subject comprising administering a particle as described herein. The particles can be administered in any appropriate pharmaceutical formulation.

The administration of the particles and compositions comprising the particles can be accomplished through any route known in the art. Routes of administration include intravenous or parenteral administration, oral administration, topical administration, transmucosal administration and transdermal administration. For intravenous or parenteral administration, i.e., injection or infusion, the composition may also contain suitable pharmaceutical diluents and carriers, such as water, saline, dextrose solutions, fructose solutions, ethanol, or oils of animal, vegetative, or synthetic origin. It may also contain preservatives, and buffers as are known in the art. When a therapeutically effective amount is administered by intravenous, cutaneous or subcutaneous injection, the solution can also contain components to adjust pH, isotonicity, stability, and the like, all of which is within the skill in the art. The pharmaceutical composition of the present invention may also contain stabilizers, preservatives, buffers, antioxidants, or other additive known to those of skill in the art.

Topical administration of an agent containing an antibiotic can be accomplished using any formulation suitable for application to the body surface, and may comprise, for example, an ointment, cream, gel, lotion, solution, paste or the like, and/or may be prepared so as to contain liposomes, micelles, and/or microspheres and/or microneedles. Preferred topical formulations herein are ointments, creams, and gels.

Ointments, as is well known in the art of pharmaceutical formulation, are semisolid preparations that are typically based on petrolatum or other petroleum derivatives. The specific ointment base to be used, as will be appreciated by those skilled in the art, is one that will provide for optimum drug delivery, and, preferably, will provide for other desired characteristics as well, e.g., emolliency or the like. As with other carriers or vehicles, an ointment base should be inert, stable, nonirritating and nonsensitizing. As explained in Remington: The Science and Practice of Pharmacy (2000), supra, ointment bases may be grouped in four classes: oleaginous bases; emulsifiable bases; emulsion bases; and water-soluble bases. Oleaginous ointment bases include, for example, vegetable oils, fats obtained from animals, and semisolid hydrocarbons obtained from petroleum. Emulsifiable ointment bases, also known as absorbent ointment bases, contain little or no water and include, for example, hydroxystearin sulfate, anhydrous lanolin and hydrophilic petrolatum. Emulsion ointment bases are either water-in-oil (W/O) emulsions or oil-in-water (O/W) emulsions, and include, for example, cetyl alcohol, glyceryl monostearate, lanolin and stearic acid. Preferred water-soluble ointment bases are prepared from polyethylene glycols of varying molecular weight (See, e.g., Remington: The Science and Practice of Pharmacy (2002), supra).

Creams, as also well known in the art, are viscous liquids or semisolid emulsions, either oil-in-water or water-in-oil. Cream bases are water-washable, and contain an oil phase, an emulsifier and an aqueous phase. The oil phase, also called the “internal” phase, is generally comprised of petrolatum and a fatty alcohol such as cetyl or stearyl alcohol. The aqueous phase usually, although not necessarily, exceeds the oil phase in volume, and generally contains a humectant. The emulsifier in a cream formulation is generally a nonionic, anionic, cationic or amphoteric surfactant.

As will be appreciated by those working in the field of pharmaceutical formulation, gels—are semisolid, suspension-type systems. Single-phase gels contain organic macromolecules distributed substantially uniformly throughout the carrier liquid, which is typically aqueous, but also, preferably, contain an alcohol and, optionally, an oil. Preferred “organic macromolecules,” i.e., gelling agents, are crosslinked acrylic acid polymers such as the “carbomer” family of polymers, e.g., carboxypolyalkylenes that may be obtained commercially under the Carbopol® trademark. Also preferred are hydrophilic polymers such as polyethylene oxides, polyoxyethylene-polyoxypropylene copolymers and polyvinylalcohol; cellulosic polymers such as hydroxypropyl cellulose, hydroxyethyl cellulose, hydroxypropyl methylcellulose, hydroxypropyl methylcellulose phthalate, and methylcellulose; gums such as tragacanth and xanthan gum; sodium alginate; and gelatin. In order to prepare a uniform gel, dispersing agents such as alcohol or glycerin can be added, or the gelling agent can be dispersed by trituration, mechanical mixing, and/or stirring.

Various additives, known to those skilled in the art, may be included in the topical formulations. For example, solubilizers may be used to solubilize certain active agents. For those drugs having an unusually low rate of permeation through the skin or mucosal tissue, it may be desirable to include a permeation enhancer in the formulation; suitable enhancers are as described elsewhere herein.

Typically, compositions for intravenous or parenteral administration comprise a suitable sterile solvent, which may be an isotonic aqueous buffer or pharmaceutically acceptable organic solvent. The compositions can also include a solubilizing agent as is known in the art if necessary. Compositions for intravenous or parenteral administration can optionally include a local anesthetic to lessen pain at the site of the injection. Generally, the ingredients are supplied either separately or mixed together in unit dosage form in a hermetically sealed container such as an ampoule or sachette. The pharmaceutical compositions for administration by injection or infusion can be dispensed, for example, with an infusion bottle containing, for example, sterile pharmaceutical grade water or saline. Where the pharmaceutical compositions are administered by injection, an ampoule of sterile water for injection, saline, or other solvent such as a pharmaceutically acceptable organic solvent can be provided so that the ingredients can be mixed prior to administration.

The duration of intravenous therapy using the pharmaceutical composition of the present invention will vary, depending on the condition being treated or ameliorated and the condition and potential idiosyncratic response of each individual mammal. The duration of each infusion is from about 1 minute to about 1 hour. The infusion can be repeated as necessary.

Systemic formulations include those designed for administration by injection, e.g., subcutaneous, intravenous, intramuscular, intrathecal or intraperitoneal injection. Useful injectable preparations include sterile suspensions, solutions or emulsions of the active compound(s) in aqueous or oily vehicles. The compositions also can contain solubilizing agents, formulating agents, such as suspending, stabilizing and/or dispersing agent. The formulations for injection can be presented in unit dosage form, e.g., in ampules or in multidose containers, and can contain added preservatives. For prophylactic administration, the compound can be administered to a patient at risk of developing one of the previously described conditions or diseases. Alternatively, prophylactic administration can be applied to avoid the onset of symptoms in a patient suffering from or formally diagnosed with the underlying condition.

The amount of compound administered will depend upon a variety of factors, including, for example, the particular indication being treated, the mode of administration, whether the desired benefit is prophylactic or therapeutic, the severity of the indication being treated and the age and weight of the patient, the bioavailability of the particular active compound, and the like. Determination of an effective dosage is well within the capabilities of those skilled in the art coupled with the general and specific examples disclosed herein.

Oral administration of the composition or vehicle can be accomplished using dosage forms including but not limited to capsules, caplets, solutions, suspensions and/or syrups. Such dosage forms are prepared using conventional methods known to those in the field of pharmaceutical formulation and described in the pertinent texts, e.g., in Remington: The Science and Practice of Pharmacy (2000), supra.

The dosage form may be a capsule, in which case the active agent-containing composition may be encapsulated in the form of a liquid. Suitable capsules may be either hard or soft, and are generally made of gelatin, starch, or a cellulosic material, with gelatin capsules preferred. Two-piece hard gelatin capsules are preferably sealed, such as with gelatin bands or the like. See, for e.g., Remington: The Science and Practice of Pharmacy (2000), supra, which describes materials and methods for preparing encapsulated pharmaceuticals.

Capsules may, if desired, be coated so as to provide for delayed release. Dosage forms with delayed release coatings may be manufactured using standard coating procedures and equipment. Such procedures are known to those skilled in the art and described in the pertinent texts (see, for e.g., Remington: The Science and Practice of Pharmacy (2000), supra). Generally, after preparation of the capsule, a delayed release coating composition is applied using a coating pan, an airless spray technique, fluidized bed coating equipment, or the like. Delayed release coating compositions comprise a polymeric material, e.g., cellulose butyrate phthalate, cellulose hydrogen phthalate, cellulose proprionate phthalate, polyvinyl acetate phthalate, cellulose acetate phthalate, cellulose acetate trimellitate, hydroxypropyl methylcellulose phthalate, hydroxypropyl methylcellulose acetate, dioxypropyl methylcellulose succinate, carboxymethyl ethylcellulose, hydroxypropyl methylcellulose acetate succinate, polymers and copolymers formed from acrylic acid, methacrylic acid, and/or esters thereof.

Sustained-release dosage forms provide for drug release over an extended time period, and may or may not be delayed release. Generally, as will be appreciated by those of ordinary skill in the art, sustained-release dosage forms are formulated by dispersing a drug within a matrix of a gradually bioerodible (hydrolyzable) material such as an insoluble plastic, a hydrophilic polymer, or a fatty compound. Insoluble plastic matrices may be comprised of, for example, polyvinyl chloride or polyethylene. Hydrophilic polymers useful for providing a sustained release coating or matrix cellulosic polymers include, without limitation: cellulosic polymers such as hydroxypropyl cellulose, hydroxyethyl cellulose, hydroxypropyl methyl cellulose, methyl cellulose, ethyl cellulose, cellulose acetate, cellulose acetate phthalate, cellulose acetate trimellitate, hydroxypropylmethyl cellulose phthalate, hydroxypropylcellulose phthalate, cellulose hexahydrophthalate, cellulose acetate hexahydrophthalate, and carboxymethylcellulose sodium; acrylic acid polymers and copolymers, preferably formed from acrylic acid, methacrylic acid, acrylic acid alkyl esters, methacrylic acid alkyl esters, and the like, e.g. copolymers of acrylic acid, methacrylic acid, methyl acrylate, ethyl acrylate, methyl methacrylate and/or ethyl methacrylate, with a terpolymer of ethyl acrylate, methyl methacrylate and trimethylammonioethyl methacrylate chloride (sold under the tradename Eudragit RS) preferred; vinyl polymers and copolymers such as polyvinyl pyrrolidone, polyvinyl acetate, polyvinylacetate phthalate, vinylacetate crotonic acid copolymer, and ethylene-vinyl acetate copolymers; zein; and shellac, ammoniated shellac, shellac-acetyl alcohol, and shellac n-butyl stearate. Fatty compounds for use as a sustained release matrix material include, but are not limited to, waxes generally (e.g., carnauba wax) and glyceryl tristearate.

In embodiments, the particles release at least about 25% of the antibiotic within the matrix of the particle within 48 hours of administration. In embodiments, the particles release at least about 50% of the antibiotic within the matrix of the particle within 48 hours of administration. In embodiments, the particles release at least about 75% of the antibiotic within the matrix of the particle within 48 hours of administration. In embodiments, the particles release at least about 90% of the antibiotic within the matrix of the particle within 48 hours of administration. In embodiments, the particles release at least about 95% of the antibiotic within the matrix of the particle within 48 hours of administration. In embodiments, the particles release from about 50% to about 100% of the antibiotic within the matrix of the particle within 48 hours of administration. In embodiments, the particles release from about 75% to about 95% of the antibiotic within the matrix of the particle within 48 hours of administration. In embodiments, the particles release from about 85% to about 90% of the antibiotic within the matrix of the particle within 48 hours of administration. In embodiments, the above % releases are within 36 hours after administration. In embodiments, the above % releases are within 24 hours after administration. In embodiments, the above % releases are within 12 hours after administration. In embodiments, the above % releases are within 6 hours after administration. In embodiments, the above % releases are within 4 hours after administration. In embodiments, the above % releases are within 2 hours after administration. In embodiments, the above % releases are within 1 hour after administration.

Transmucosal administration of an agent containing an antibiotic can be accomplished using any type of formulation or dosage unit suitable for application to mucosal tissue. For example, the particles containing an antibiotic can be administered to the buccal mucosa in an adhesive patch, sublingually or lingually as a cream, ointment, or paste, nasally as droplets or a nasal spray, or by inhalation of an aerosol formulation or a non-aerosol liquid formulation.

Preferred buccal dosage forms will typically comprise a therapeutically effective amount of an antibiotic and a bioerodible (hydrolyzable) polymeric carrier that may also serve to adhere the dosage form to the buccal mucosa. The buccal dosage unit is fabricated so as to erode over a predetermined time period, wherein drug delivery is provided essentially throughout. The time period is typically in the range of from about 1 hour to about 72 hours. Preferred buccal delivery preferably occurs over a time period of from about 2 hours to about 24 hours. Buccal drug delivery for short-term use should preferably occur over a time period of from about 2 hours to about 8 hours, more preferably over a time period of from about 3 hours to about 4 hours. As needed buccal drug delivery preferably will occur over a time period of from about 1 hour to about 12 hours, more preferably from about 2 hours to about 8 hours, most preferably from about 3 hours to about 6 hours. Sustained buccal drug delivery will preferably occur over a time period of from about 6 hours to about 72 hours, more preferably from about 12 hours to about 48 hours, most preferably from about 24 hours to about 48 hours. Buccal drug delivery, as will be appreciated by those skilled in the art, avoids the disadvantages encountered with oral drug administration, e.g., slow absorption, degradation of the active agent by fluids present in the gastrointestinal tract and/or first-pass inactivation in the liver.

The “therapeutically effective amount” of an agent in the buccal dosage unit will of course depend on the potency and the intended dosage, which, in turn, is dependent on the particular individual undergoing treatment, the specific indication, and the like. The buccal dosage unit will generally contain from about 1.0 wt. % to about 60 wt. % active agent, preferably on the order of from about 1 wt. % to about 30 wt. % active agent. With regard to the bioerodible (hydrolyzable) polymeric carrier, it will be appreciated that virtually any such carrier can be used, so long as the desired drug release profile is not compromised, and the carrier is compatible with any other components of the buccal dosage unit. Generally, the polymeric carrier comprises a hydrophilic (water-soluble and water-swellable) polymer that adheres to the wet surface of the buccal mucosa. Examples of polymeric carriers useful herein include acrylic acid polymers and co, e.g., those known as “carbomers” (Carbopol®, which may be obtained from B. F. Goodrich, is one such polymer). Other suitable polymers include, but are not limited to: hydrolyzed polyvinylalcohol; polyethylene oxides (e.g., Sentry Polyox® water soluble resins, available from Union Carbide); polyacrylates (e.g., Gantrez®, which may be obtained from GAF); vinyl polymers and copolymers; polyvinylpyrrolidone; dextran; guar gum; pectins; starches; and cellulosic polymers such as hydroxypropyl methylcellulose, (e.g., Methocel®, which may be obtained from the Dow Chemical Company), hydroxypropyl cellulose (e.g., Klucel®, which may also be obtained from Dow), hydroxypropyl cellulose ethers (see, e.g., U.S. Pat. No. 4,704,285 to Alderman), hydroxyethyl cellulose, carboxymethyl cellulose, sodium carboxymethyl cellulose, methyl cellulose, ethyl cellulose, cellulose acetate phthalate, cellulose acetate butyrate, and the like.

Other components may also be incorporated into the buccal dosage forms described herein. The additional components include, but are not limited to, disintegrants, diluents, binders, lubricants, flavoring, colorants, preservatives, and the like. Examples of disintegrants that may be used include, but are not limited to, cross-linked polyvinylpyrrolidones, such as crospovidone (e.g., Polyplasdone® XL, which may be obtained from GAF), cross-linked carboxylic methylcelluloses, such as croscarmelose (e.g., Ac-di-Sol®, which may be obtained from FMC), alginic acid, and sodium carboxymethyl starches (e.g., Explotab®, which may be obtained from Edward Medell Co., Inc.), methylcellulose, agar bentonite and alginic acid. Suitable diluents are those which are generally useful in pharmaceutical formulations prepared using compression techniques, e.g., dicalcium phosphate dihydrate (e.g., Di-Tab®, which may be obtained from Stauffer), sugars that have been processed by cocrystallization with dextrin (e.g., co-crystallized sucrose and dextrin such as Di-Pak®, which may be obtained from Amstar), calcium phosphate, cellulose, kaolin, mannitol, sodium chloride, dry starch, powdered sugar and the like. Binders, if used, are those that enhance adhesion. Examples of such binders include, but are not limited to, starch, gelatin and sugars such as sucrose, dextrose, molasses, and lactose. Particularly preferred lubricants are stearates and stearic acid, and an optimal lubricant is magnesium stearate.

Sublingual and lingual dosage forms include creams, ointments and pastes. The cream, ointment or paste for sublingual or lingual delivery comprises a therapeutically effective amount of the selected active agent and one or more conventional nontoxic carriers suitable for sublingual or lingual drug administration. The sublingual and lingual dosage forms of the present invention can be manufactured using conventional processes. The sublingual and lingual dosage units are fabricated to disintegrate rapidly. The time period for complete disintegration of the dosage unit is typically in the range of from about 10 seconds to about 30 minutes, and optimally is less than 5 minutes.

Other components may also be incorporated into the sublingual and lingual dosage forms described herein. The additional components include, but are not limited to binders, disintegrants, wetting agents, lubricants, and the like. Examples of binders that may be used include water, ethanol, polyvinylpyrrolidone; starch solution gelatin solution, and the like. Suitable disintegrants include dry starch, calcium carbonate, polyoxyethylene sorbitan fatty acid esters, sodium lauryl sulfate, stearic monoglyceride, lactose, and the like. Wetting agents, if used, include glycerin, starches, and the like. Particularly preferred lubricants are stearates and polyethylene glycol. Additional components that may be incorporated into sublingual and lingual dosage forms are known, or will be apparent, to those skilled in this art (See, e.g., Remington: The Science and Practice of Pharmacy (2000), supra).

Other preferred compositions for sublingual administration include, for example, a bioadhesive; a spray, paint, or swab applied to the tongue; or the like. Increased residence time increases the likelihood that the administered invention can be absorbed by the mucosal tissue.

Transdermal administration of a particle containing an antibiotic through the skin or mucosal tissue can be accomplished using conventional transdermal drug delivery systems, wherein the agent is contained within a laminated structure (typically referred to as a transdermal “patch”) that serves as a drug delivery device to be affixed to the skin.

Transdermal drug delivery may involve passive diffusion or it may be facilitated using electrotransport, e.g., iontophoresis. In a typical transdermal “patch,” the drug composition is contained in a layer, or “reservoir,” underlying an upper backing layer. The laminated structure may contain a single reservoir, or it may contain multiple reservoirs. In one type of patch, referred to as a “monolithic” system, the reservoir is comprised of a polymeric matrix of a pharmaceutically acceptable contact adhesive material that serves to affix the system to the skin during drug delivery. Examples of suitable skin contact adhesive materials include, but are not limited to, polyethylenes, polysiloxanes, polyisobutylenes, polyacrylates, polyurethanes, and the like. Alternatively, the drug-containing reservoir and skin contact adhesive are separate and distinct layers, with the adhesive underlying the reservoir which, in this case, may be either a polymeric matrix as described above, or it may be a liquid or hydrogel reservoir, or may take some other form.

The backing layer in these laminates, which serves as the upper surface of the device, functions as the primary structural element of the laminated structure and provides the device with much of its flexibility. The material selected for the backing material should be selected so that it is substantially impermeable to the active agent and any other materials that are present, the backing is preferably made of a sheet or film of a flexible elastomeric material. Examples of polymers that are suitable for the backing layer include polyethylene, polypropylene, polyesters, and the like.

During storage and prior to use, the laminated structure includes a release liner. Immediately prior to use, this layer is removed from the device to expose the basal surface thereof, either the drug reservoir or a separate contact adhesive layer, so that the system may be affixed to the skin. The release liner should be made from a drug/vehicle impermeable material.

Transdermal drug delivery systems may in addition contain a skin permeation enhancer. That is, because the inherent permeability of the skin to some drugs may be too low to allow therapeutic levels of the drug to pass through a reasonably sized area of unbroken skin, it is necessary to coadminister a skin permeation enhancer with such drugs. Suitable enhancers are well known in the art and include, for example, those enhancers listed below in transmucosal compositions.

Formulations can comprise one or more anesthetics. Patient discomfort or phlebitis and the like can be managed using anesthetic at the site of injection. If used, the anesthetic can be administered separately or as a component of the composition. One or more anesthetics, if present in the composition, is selected from the group consisting of lignocaine, bupivacaine, dibucaine, procaine, chloroprocaine, prilocaine, mepivacaine, etidocaine, tetracaine, lidocaine and xylocaine, and salts, derivatives or mixtures thereof.

In particular, aerosolized medicaments are used to deliver particles to the lungs by having the patient inhale the aerosol through a tube and/or mouthpiece coupled to the aerosol generator. By inhaling the aerosolized medicament, the patient can quickly receive a dose of medicament in the lungs. In this way, the particles are delivered in a manner that can be the most efficient for licensing immunity. Aerosols of solid particles comprising the antibiotic may be produced with any solid particulate medicament aerosol generator. Aerosol generators for administering solid particulate medicaments to a subject produce particles which are respirable, as explained above, and generate a volume of aerosol containing a predetermined metered dose of a medicament at a rate suitable for human administration. One illustrative type of solid particulate aerosol generator is an insufflator. Suitable formulations for administration by insufflation include finely comminuted powders which may be delivered by means of an insufflator or taken into the nasal cavity in the manner of a snuff. In the insufflator, the powder (e.g., a metered dose thereof effective to carry out the treatments described herein) is contained in capsules or cartridges, typically made of gelatin or plastic, which are either pierced or opened in situ and the powder delivered by air drawn through the device upon inhalation or by means of a manually-operated pump. The powder employed in the insufflator consists either solely of the active ingredient or of a powder blend comprising the anti-malarial compound, a suitable powder diluent, such as lactose, and an optional surfactant. A second type of illustrative aerosol generator comprises a metered dose inhaler. Metered dose inhalers are pressurized aerosol dispensers, typically containing a suspension or solution formulation of the anti-malarial compound in a liquified propellant. During use these devices discharge the formulation through a valve, adapted to deliver a metered volume, from 10 to 22 microliters to produce a fine particle spray containing the anti-malarial compound.

Suitable propellants include certain chlorofluorocarbon (compounds, for example, dichlorodifluoromethane, trichlorofluoromethane, dichlorotetrafluoroethane and mixtures thereof. The formulation may additionally contain one or more co-solvents, for example, ethanol, surfactants, such as oleic acid or sorbitan trioleate, antioxidants and suitable flavoring agents. Any propellant may be used in carrying out the present invention, including both chlorofluorocarbon-containing propellants and non-chlorofluorocarbon-containing propellants. Fluorocarbon aerosol propellants that may be employed in carrying out the present invention including fluorocarbon propellants in which all hydrogen are replaced with fluorine, chlorofluorocarbon propellants in which all hydrogens are replaced with chlorine and at least one fluorine, hydrogen-containing fluorocarbon propellants, and hydrogen-containing chlorofluorocarbon propellants. A stabilizer such as a fluoropolymer may optionally be included in formulations of fluorocarbon propellants, such as described in U.S. Pat. No. 5,376,359 to Johnson.

In pulmonary delivery in particular, therapeutics must circumvent the lung's particle clearance mechanisms such as mucociliary transport, phagocytosis by macrophages and rapid absorption of drug molecules into the systemic circulation. Mucociliary clearance can be reduced by avoiding particle deposition in the tracheobronchial region which contains the cilia and goblets cells that make up the mucociliary escalator. Upon delivery to the pulmonary region, particles can be rapidly cleared by alveolar macrophages.

The subject matter described herein includes the following embodiments:

1. A pharmaceutical composition comprising:
a plurality of particles wherein each particle of the plurality comprises poly(D,L-lactide-co-glycolide) (PLGA), a cationic lipid or a pharmaceutically acceptable salt thereof, and an antibiotic.
   2. The pharmaceutical composition of embodiment 1, wherein the poly(D,L-lactide-co-glycolide) comprises a molar ratio for D,L-lactide:glycolide of approximately 50:50.
   3. The pharmaceutical composition of any above embodiment, wherein the molecular weight average, Mw, of the poly(D,L-lactide-co-glycolide) is between about 16 kDa and about 54 kDa.
   4. The pharmaceutical composition of any above embodiment, wherein the molecular weight average, Mw, of the poly(D,L-lactide-co-glycolide) is between about 38 kDa and about 54 kDa.
   5. The pharmaceutical composition of any above embodiment, wherein the molecular weight average, Mw, of the poly(D,L-lactide-co-glycolide) is between about 24 kDa and about 38 kDa.
   6. The pharmaceutical composition of any above embodiment, wherein the molecular weight average, Mw, of the poly(D,L-lactide-co-glycolide) is about 36 kDa.
   7. The pharmaceutical composition of any above embodiment, wherein the molecular weight average, Mw, of the poly(D,L-lactide-co-glycolide) is about 25 kDa.
   8. The pharmaceutical composition of any above embodiment, wherein the cationic lipid comprises 1,2-dioleoyloxy-3-(trimethylammonio) propane (DOTAP) and/or a pharmaceutically acceptable salt thereof.
   9. The pharmaceutical composition of any above embodiment, wherein the antibiotic comprises levofloxacin and/or a pharmaceutically acceptable salt thereof.
   10. The pharmaceutical composition of any above embodiment, wherein the poly(D,L-lactide-co-glycolide) comprises from about 80 wt % to about 99 wt % of the composition.
   11. The pharmaceutical composition of any above embodiment, wherein the cationic lipid comprises about 15 wt % or less of the composition, about 10 wt % or less of the composition, or about 5 wt % or less of the composition.
   12. The pharmaceutical composition of any above embodiment, wherein the antibiotic comprises about 20 wt % or less of the composition, about 18 wt % or less of the composition, about 15 wt % or less of the composition, about 13.5 wt % or less of the composition, or about 12 wt % or less of the composition.
   13. The pharmaceutical composition of any above embodiment, wherein each particle of the plurality comprises a non-spherical engineered shape defined by at least two substantially planar surfaces and a maximum cross-sectional dimension of less than about 10 μm, and further wherein each particle is substantially congruent relative to another particle of the plurality of particles.
   14. The pharmaceutical composition of any above embodiment, wherein the antibiotic is dispersed substantially throughout the particle.
   15. The pharmaceutical composition of any above embodiment, wherein the non-spherical engineered shape comprises a cylinder.
   16. The pharmaceutical composition of any above embodiment, wherein the cylinder size is selected from the group consisting of approximately d=1 μm×h=1 μm, approximately d=2 μm×h=0.6 μm, and approximately d=3 μm×1 μm; wherein d represents the diameter and h represents the height of the cylinder.
   17. The pharmaceutical composition of any above embodiment, wherein the poly(D,L-lactide-co-glycolide) has an average molecular weight of about 25 kDa, the cationic lipid is 1,2-dioleoyloxy-3-(trimethylammonio) propane (DOTAP) and/or a pharmaceutically acceptable salt thereof and comprises about 5 wt % of the composition, and the antibiotic is levofloxacin and comprises about 13.5 wt % of the composition.
   18. A pharmaceutical composition for administration to treat or prevent urinary tract infection (UTI) comprising:
a plurality of particles wherein each particle of the plurality comprises poly(D,L-lactide-co-glycolide) (PLGA), a cationic lipid or a pharmaceutically acceptable salt thereof, and an antibiotic.
   19. The pharmaceutical composition of embodiment 18, wherein administration is via instillation or intravenously.
   20. The pharmaceutical composition of embodiment 18, wherein the poly(D,L-lactide-co-glycolide) has an average molecular weight of about 25 kDa, the cationic lipid is 1,2-dioleoyloxy-3-(trimethylammonio) propane (DOTAP) and/or a pharmaceutically acceptable salt thereof and comprises about 5 wt % of the composition, and the antibiotic is levofloxacin and comprises about 13.5 wt % of the composition.
   21. A method of treating a UTI in a subject in need thereof comprising, administering to said subject a therapeutically effective amount of a composition in any one of the above embodiments.
   22. The method of embodiment 21, wherein said administering said particle provides a therapeutic level of antibiotic for up to 24 hours.
   22. The method of embodiment 21, wherein said subject has a neurogenic bladder.
   23. The method of embodiment 21, wherein said subject has a spinal cord injury.

The subject matter described herein includes the following further embodiments:

In an embodiment, a method of preventing or reducing severity of a urinary tract infection in a subject in need thereof, the method comprising:
administering a therapeutically effective amount of a plurality of particles, wherein each particle of the plurality comprises:
   a biocompatible matrix comprising:
      poly(D,L-lactide-co-glycolide),
      a cationic agent and/or a pharmaceutically acceptable salt thereof;
   and
      an antibiotic and/or pharmaceutically acceptable salt thereof,
      wherein the antibiotic is dispersed substantially throughout the biocompatible matrix; and
   a non-spherical three-dimensional engineered shape comprising:
      at least two substantially planar surfaces, and
      in cross-section, a maximum dimension of less than about 10 μm;
wherein administration provides protection to the urinary bladder from 104 kanamycin resistance-marked E. coli UTI189 for at least eight hours.

As in any embodiment above, a method wherein the administration provides protection to the kidneys from 104 kanamycin resistance-marked E. coli UTI189 for at least eighteen hours.

As in any embodiment above, a method wherein administration comprises instilling the plurality of particles into the bladder of a subject and expelling urine from the subject approximately one hour after instillation.

As in any embodiment above, a method wherein the poly(D,L-lactide-co-glycolide) comprises a molecular weight average (Mw) of about 16 kDa to about 54 kDa.

As in any embodiment above, a method wherein the poly(D,L-lactide-co-glycolide) comprises a molecular weight average (Mw) of about 24 kDa to about 38 kDa.

As in any embodiment above, a method wherein the poly(D,L-lactide-co-glycolide) comprises a molecular weight average (Mw) of about 38 kDa to about 54 kDa.

As in any embodiment above, a method wherein the poly(D,L-lactide-co-glycolide) comprises a molecular weight average (Mw) of about 25 kDa.

As in any embodiment above, a method wherein the poly(D,L-lactide-co-glycolide) comprises a molecular weight average (Mw) of about 6 kDa.

As in any embodiment above, a method wherein the poly(D,L-lactide-co-glycolide) comprises a molar ratio of D,L-lactide:glycolide of about 50:50.

As in any embodiment above, a method wherein the cationic agent and/or pharmaceutically acceptable salt thereof is selected from the group consisting of cationic lipids, cationic polymers, cationic lipidoids, and cationic agents containing a portion having a positive charge in aqueous solutions at neutral pH.

As in any embodiment above, a method wherein the antibiotic and/or pharmaceutically acceptable salt thereof comprises a quinolone or a fluoroquinolone.

As in any embodiment above, a method wherein the antibiotic and/or pharmaceutically acceptable salt thereof comprises levofloxacin.

As in any embodiment above, a method wherein the antibiotic and/or pharmaceutically acceptable salt thereof comprises levofloxacin and the levofloxacin comprises up to about 13.5 wt % of the particle.

As in any embodiment above, a method wherein the non-spherical three-dimensional engineered shape comprises a rod.

As in any embodiment above, a method wherein the rod size is selected from approximately d=1 μm×h=1 μm, d=2 μm×h=0.6 μm, and d=3 μm×h=1 μm; wherein d represents the diameter and h represents the height of the rod.

As in any embodiment above, a method wherein the shape in cross-section is a rectangle, circle, and/or ellipsoid.

As in any embodiment above, a method wherein administration is via infiltration.

As in any embodiment above, a method wherein administration provides topical, intracystic prophylaxis against urinary tract infection for subjects having a neurogenic bladder.

In an embodiment, a method of preventing or reducing severity of a urinary tract infection in a subject in need thereof, the method comprising:
administering a therapeutically effective amount of a plurality of particles, wherein each particle of the plurality comprises:
   a biocompatible matrix comprising:
      poly(D,L-lactide-co-glycolide) comprising a molecular weight average (Mw) of about 16 kDa to about 54 kDa,
      1,2-dioleoyl-3-trimethylammonium-propane (DOTAP) and/or a pharmaceutically acceptable salt thereof, wherein a pre-particle solution used to fabricate the particle comprises about 0 wt % to about 5 wt % DOTAP; and
   levofloxacin and/or pharmaceutically acceptable salt thereof, wherein:
      levofloxacin comprises about 3.5 wt % to about 13.5 wt % of the particle and
      the antibiotic is dispersed substantially throughout the biocompatible matrix; and
   a non-spherical three-dimensional engineered shape comprising:
      at least two substantially planar surfaces, and
      in cross-section, a maximum dimension of less than about 10 μm.

As in any embodiment above, a method wherein administration provides protection to the urinary bladder from 104 kanamycin resistance-marked E. coli UT1189 for at least eight hours.

The present subject matter is further described herein by the following non-limiting examples which further illustrate the invention, and are not intended, nor should they be interpreted to, limit the scope of the invention.

### EXAMPLES

### Methods and Materials:

Poly(D,L-lactide-co-glycolide)s (PLGA, lactide:glycolide 50:50, Mw 16-54 K) were purchased from Sigma-Aldrich (St. Louis, Mo.) and Evonik Industries (Tippecanoe, Ind.). 1,2-dioleoyl-3-trimethylammonium-propane (chloride salt) (DOTAP) was purchased from Avanti Polar Lipids (Alabaster, Ala.). Chloroform, acetonitrile and water for high performance liquid chromatography (HPLC) were purchased from Fisher Scientific (Waltham, Mass.). Poly(ethylene terephthalate) (PET) sheets (6″ width) were purchased from KRS plastics (Tabor City, N.C.). Liquidia Technologies, Inc. (Research Triangle Park, N.C.) provided PRINT® molds under license. All other chemicals were purchased from Sigma-Aldrich. Sterile ultrapure water was used throughout the study.

Tissue culture-based microbial inhibition assays-Bladder epithelial cell lines 5637 and UROTSA have been previously described [27,28]. Tissue culture cells were growth in RPMI 1640 media to 80% confluence and lifted using trypsin-EDTA (Sigma-Aldrich). Twenty four well plates (Genesee Scientific) were seeded with 5637 cells and grown for 2 days at 37° C., 5% CO2 prior to infections. Where appropriate to the assay conditions, unencapsulated (“free”) levofloxacin was added to the wells one hour prior to infection and at the same concentration as wells treated with PRINT-levofloxacin particles. Empty levofloxacin particles (referred to as “BLANK”) were added at the same weight/volume concentration as used in the comparable PRINT-levofloxacin wells. At the time of infection, 100 μl colony-forming units of bacteria were introduced into the appropriate assay wells at an approximate multiplicity of infection (MOI) of 10:1. Infected tissue culture cells were incubated at 37° C., 5% CO02 until the assay endpoint as described in the text. Infected and uninfected epithelial monolayers were harvested by being scraped off into 0.1% Triton X100 sterile buffer. Dilutions of the harvested and lysed epithelial cells were plated on LB agar plates to enumerate the viable bacteria. Plates were incubated overnight at 37° C. prior to counting.

### Example 1: Particle Fabrication

Levofloxacin PRINT particles were fabricated as described above and as reported in the literature with modifications [23]. Generally, chloroform was used as solvent for the pre-particle solution, which was composed by 100 mg PLGA, 0-50 mg levofloxacin and 0-7.5 mg DOTAP. A thin film was made by evaporating 130 μL of pre-particle solution on a PET sheet followed by the particle transferring and harvesting processes as previously described [23]. The particles were purified and concentrated by centrifugation and were flash frozen in 0.1% wt/wt % polyvinyl alcohol (PVOH, Mw 2000) aqueous solution.

The particle surface charge was measured by dynamic light scattering in pure water at room temperature (Malvern Instruments Nano-ZS). The particle size and morphology were checked by SEM imaging (Hitachi model S-4700) as reported previously [23].

### Example 2: Levofloxacin Loading and In Vitro Release

Levofloxacin loading efficiency in the PLGA-PRINT particles was determined by HPLC. (Agilent Technologies Series 1200 HPLC with a C18 reverse phase column). For HPLC analysis, 100 μL particle sample was dissolved in 900 μL acetonitrile and mixed by vortexing for 1 hour. A linear gradient from 100% water to 100% acetonitrile was run over 15 min. Then 100% acetonitrile was run for 5 min. The flow rate was 1 mL/min and detection was at 210 nm and 290 nm for PLGA and levofloxacin respectively.

For the drug release assay, 100 μL of particle solution (100 mg of particles/mL) was placed in a mini dialysis unit with a 20 K MW cutoff and dialysed against a gently stirred 1 L bath of PBS (10 mM phosphate buffer, 138 mM NaCl and 2.7 mM KCl; pH 7.4) at 37° C. Three dialysis units were used for each time point. Then, the particle solution in each dialysis unit was removed and centrifuged to pellet the PLGA-PRINT particles. The pellet was then analyzed for the amount of remaining levofloxacin using HPLC as described above.

### Example 3: Bacteria and Growth Conditions

E. coli strain UT189 (O18:K1:H7) a human cystitis strain [24], was used in all of the experiments. Bacteria were routinely grown in Luria broth (LB) for in vitro studies and in preparation for tissue culture infections. Overnight cultures were grown shaking at 250 RPM at 37° C. and then 1:100 subcultures for an additional 18 hours growth without shaking (static) at 37° C. [25,26]. In preparation for infection, the bacteria were centrifuged at 1,250×g for 10 minutes and resuspended in sterile phosphate buffered saline (PBS) to the concentration required for the respective experiments.

### Example 4: In Vitro Killing of E. coli by Free and PRINT-Encapsulated Levofloxacin

For the determination of the minimal bactericidal concentration of free levofloxacin or PRINT encapsulated levofloxacin in liquid culture, 1×103 colony-forming units (CFU) per milliliter of E. coli UT189 were mixed with doses of free levofloxacin (Sigma-Aldrich) or PRINT-encapsulated levofloxacin and shaken for 2 hours at 37° C. CFU were enumerated by spreading 20 μl on a LB plate followed by growth at 37° C. overnight prior to counting. The upper limit of CFU per plate was 200.

For the measurement of the zones of clearing produced by free levofloxacin or PRINT encapsulated levofloxacin, bacteria were spread over the surface of an LB agar plate and allowed to dry. Over the top of the bacterial lawn, a 7 mm paper disk was positioned and spotted with 10 μl of phosphate buffered saline (PBS), PBS with levofloxacin (0-5 μg/ml), or PBS with PRINT-levofloxacin (equal to 0-5 μg levofloxacin, based on loading concentration in the PRINT particles). The plates were incubated upright at 37° C. overnight. The diameter of the zones of clearing around each disk was measured. The assay was repeated 3 times on independent days.

### Example 5: Rat Spinal Cord Injury UTI Infection Model

As described previously, female Sprague-Dawley rats (Harlan; 175-200 gm) underwent controlled surgical T10 spinal cord transection to model spinal cord injury and produce a neurogenic bladder phenotype [29]. All animals received daily surgical site and urinary tract antibiotic prophylaxis (enrofloxacin 5 mg/kg subcutaneous; Bayer) twice daily for 8 days postoperatively. Postoperative pain was relieved using daily carprofen (5 mg/kg subcutaneous; Pfizer) for the first 4 days after surgery. Due to the spinal cord injury and subsequent neurogenic bladder phenotypes, animals were routinely monitored and creded to relieve urinary retention each 12 hours. Animals had free access to food and water, which was placed at ground level to accommodate the hind leg paralysis due to the spinal cord injury.

In preparation for the administration of free or PRINT encapsulated levofloxacin, SCI rats underwent isoflurane anesthesia, and the hair surrounding the urethral meatus and perineum were trimmed with electrical clippers. The shaved area was disinfected with 3 rounds of alternating povidone-iodine and 70% ethanol. Following bladder emptying by gentle crede, an angiocath (22 guage), lubricated with Surgilube and connected to a 1 ml tuberculin syringe containing the control or experimental drug, was inserted the full length through the urethral meatus and into the bladder. Control and experimental drugs were slowly administered by a manual push. After full administration, the syringe and catheter were held in place for 30 seconds followed by removal of the catheter and recovery of the animals.

Two days prior to infection, an overnight shaking culture of E. coli UT189 was prepared, grown in LB broth with 50 μg kanamycin in the 37° C. incubator. One day prior to infection, a subculture was prepared by diluting the initial culture 1:100 into fresh LB broth with 50 μg/ml of kanamycin and incubating static for 18 hours in 37° C. room. On the day of infection the bacteria were pelleted and resuspended to an OD600=0.80 in sterile PBS (˜109 CFU/ml). The precise concentration of bacteria was determined through plating dilutions onto LB media.

Infections were performed as previously described [29,30]. Animals were administered 5×104 CFU of E. coli UT189 in 100 μl of PBS. After slow administration of the inoculum through transurethral catheterization, the catheter was held in place for 30 seconds to ensure good distribution of bacteria in the bladder. At the designated endpoints, rats were euthanized using CO2. Organs of interest were harvested using sterile technique and homogenized in 0.02% Triton X100 in PBS. Dilutions of the homogenates were plated on LB and MacConkey agar containing kanamycin (50 μg/ml). Plates were grown at 37° C. overnight prior to counting the CFU.

### Example 6: Particle Fabrication and Characterization

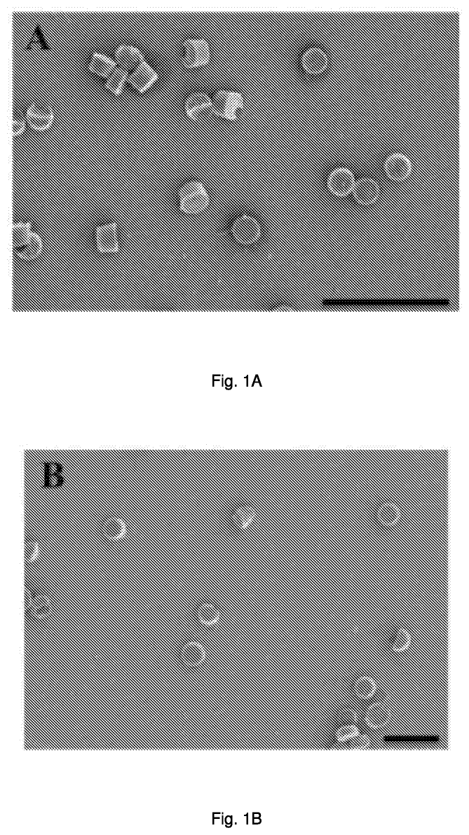

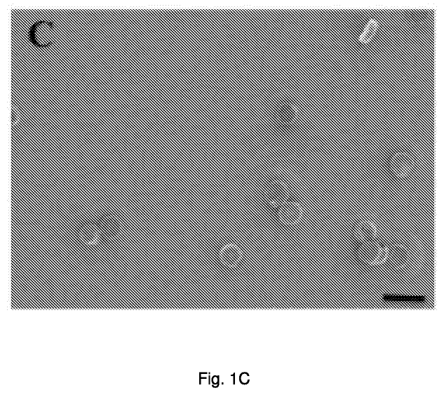

We engineered multiple formulations of PRINT encapsulated levofloxacin to determine the extent to which size, shape, and physical parameters impacted the relative effectiveness to protect cultured bladder epithelial cells from E. coli infection. Particles were manufactured with 3 dimensions: d=1 μm, h=1 μm (1×1 μm); d=2 μm, h=0.6 μm (2×0.6 μm) and d=3 μm, h=1 μm (3×1 μm). In addition to controlling particle size, we also explored different formulations to tune drug loading efficiency and surface charge, which may have great impact on drug delivery [20]. We synthesized calibration quality, monodisperse PLGA PRINT particles with a narrow polydispersity index (PDI) (FIGS. 1A-C). Drug loading efficiency differed minimally between particle batches and drug loading could be controlled by changing the particle size and the molecular weight of PLGA (Table 2). The combination of a larger PLGA-PRINT particle size and lower molecular weight PLGA matrix were used to increase the amount of encapsulated levofloxacin.

TABLE 2
Characterization data of the Levofloxacin loaded
PLGA-PRINT particles

| | | | Drug | Zeta |
|---|---|---|---|---|
| Sample | Matrix | Size | Loading efficiency | (mV) |
| D1 | PLGA, Mw 38-54 K | 1 × 1 μm | Levofloxacin | +5.5 |
| | 1.5% DOTAP | | 6.5% | |
| D2 | PLGA, Mw 24-38 K | 1 × 1 μm | Levofloxacin | +31 |
| | 2% DOTAP | | 8.5% | |
| B1 | PLGA, Mw 38-54 K | 1 × 1 μm | Blank | +23.4 |
| | 1.5% DOTAP | | | |
| B2 | PLGA, Mw 24-38 K | 1 × 1 μm | Blank | +23.8 |
| | 1.5% DOTAP | | | |
| D3 | PLGA, Mw 36 K | 1 × 1 μm | Levofloxacin | +16.5 |
| | 3% DOTAP | | 5.7% | |
| D4 | PLGA, Mw 36 K | 1 × 1 μm | Levofloxacin | −25 |
| | 0% DOTAP | | 3.5% | |
| D5 | PLGA, Mw 25 K | 1 × 1 μm | Levofloxacin | +39.5 |
| | 4% DOTAP | | 7.6% | |
| D6 | PLGA, Mw 25 K | 2 × 0.6 μm | Levofloxacin | +30 |
| | 5% DOTAP | | 10.5% | |
| D7 | PLGA, Mw 25 K | 3 × 1 μm | Levofloxacin | +4 |
| | 4% DOTAP | | 13.5% | |
| D8 | PLGA, Mw 25 K | 1 × 1 μm | Levofloxacin | +37.3 |
| | 5% DOTAP | | 4~8% | |
| | Quantum Dot | | | |
| B3 | PLGA, Mw 25 K | 1 × 1 μm | Blank | +26.7 |
| | 5% DOTAP | | | |
| B4 | PLGA, Mw 36 K | 1 × 1 μm | Blank | +36.6 |
| | 5% DOTAP | | | |

Because of the intrinsic amphipathic property of levofloxacin, it has a tendency to rapidly dissolve in water once the particles are re-suspended in aqueous solution, which significantly reduces drug retention in the particles. As a result, more than 95% of the loaded drug was released in one hour under the in vitro drug-release test conditions. Formula D4 (Table 2) provides an example of the zeta potential of an unmodified levofloxacin-loaded particle, which is highly negatively charged due to the carboxyl group on the drug. Since the goal was to develop formulations that were capable of intracellular drug delivery, we posited that cationic PRINT Levofloxacin particles would provide enhanced cellular uptake and, subsequently, enhanced therapeutic efficiency.

### Results:

In vitro comparison between unencapsulated, soluble levofloxacin and PRINT levofloxacin encapsulation inhibition of E. coli.

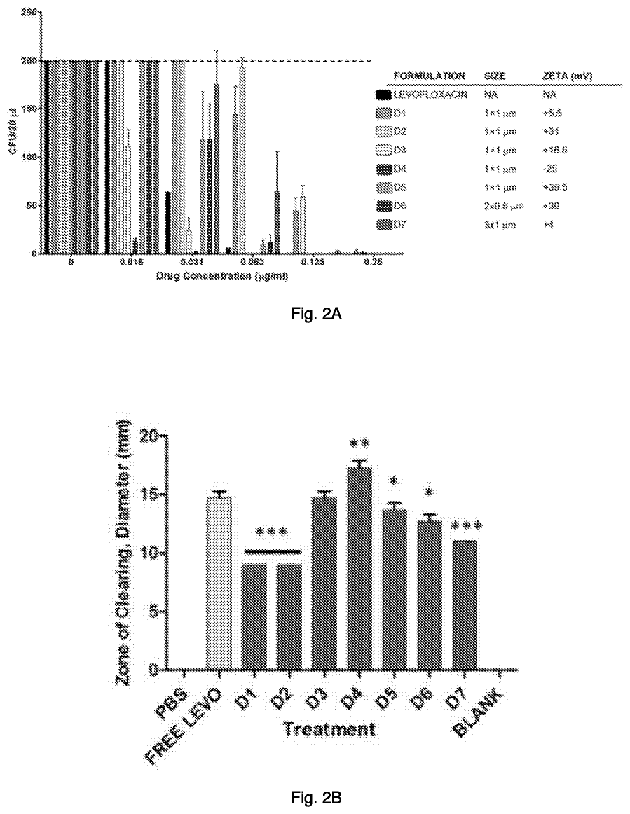

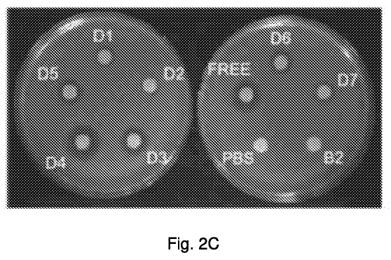

Comparison of the effectiveness of the PRINT-Levofloxacin in inhibiting bacterial growth in liquid and solid media was made. FIG. 2A shows a comparison of the growth of E. coli UT189 in liquid broth in the presence of increasing concentrations of free levofloxacin or PRINT-Levofloxacin formulations. For the formulations D1, D2, D6, and D7, encapsulated levofloxacin had higher minimal inhibitory concentrations compared to free levofloxacin (P=<0.0001 by ANOVA). Formulation D5 was only slightly less inhibitory than free levofloxacin at the concentration of 0.031 μg/ml (P<0.001), but otherwise was equivalent at matched concentrations of the free drug. Formulations D3 and D4, known to contain free and PRINT-encapsulated levofloxacin (Table 2), were each more inhibitory than free levofloxacin at concentrations between 0.016-0.063 μg/ml (P<0.0001).

Comparison of PRINT-Levofloxacin formulations with free levofloxacin in a disk diffusion assay (FIGS. 2B-C) was made. Disks were laid over a lawn of E. coli UT189 and spotted with a 5 μg/ml solution of free levofloxacin or a comparable concentration of a PRINT-Levofloxacin formulation (based on total antibiotic) in 3 independent trials. Free levofloxacin produced a large zone of clearing that was comparable to formulation D3. D4 produced a significantly larger zone; however that formulation was known to contain free levofloxacin (Table 2), which likely contributed to the high drug diffusion and thus larger zone of clearance. In contrast, D5-7 had reduced zones of clearance as did D1-2. No relationship between the zone of clearing and particle size, shape, or PLGA composition was observed. A correlation between percent weight/volume loading of levofloxacin and the zone of clearing was notable but not significant (R2=−0.52; P=0.23). Although the observed differences in the liquid and solid assay inhibitory breakpoints are not likely clinically significant in comparison to the PRINT-Levofloxacin formulations and free drug, these data suggest different kinetics of drug exposure to the bacteria and less diffusion in the microenvironment.

PRINT-levofloxacin is superior to free levofloxacin in preventing E. coli persistence in cultured bladder epithelial cells and is non-toxic. Immortalized bladder epithelial cells in tissue culture have provided an excellent model for studying the invasion of the uroepithelium by uropathogens like E. coli [31-33]. This model was employed to determine the extent to which PRINT-Levofloxacin inhibits intracellular infection and persistence of E. coli in the bladder epithelium in comparison to free levofloxacin. Bladder cell line 5637 [34,35] was pretreated with one of the PRINT-Levofloxacin formulas (Table 2) or controls that included media alone, free levofloxacin, or empty dimension and composition-matched PRINT particles. Unbound particles or free drug were gently washed from the cells and followed by infection with E. coli UT189 for 1 hour to allow invasion. Unbound bacteria were removed by gentle washing, and the cultured cells were incubated for an additional 4 hours after which time the epithelial cells were lifted, lysed, and plated to count bacteria.

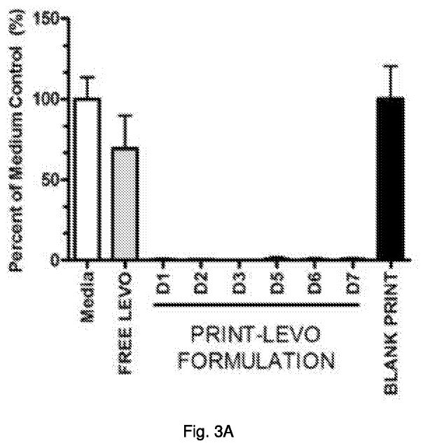

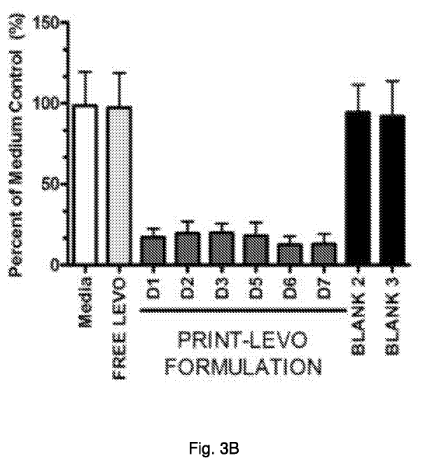

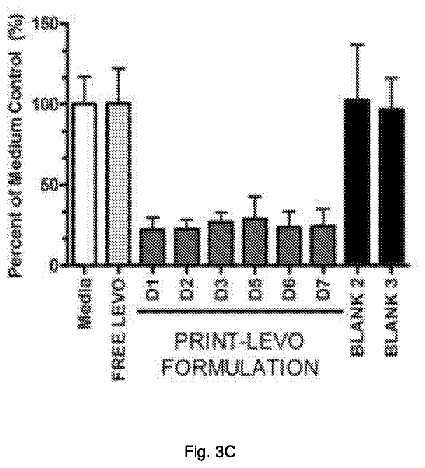

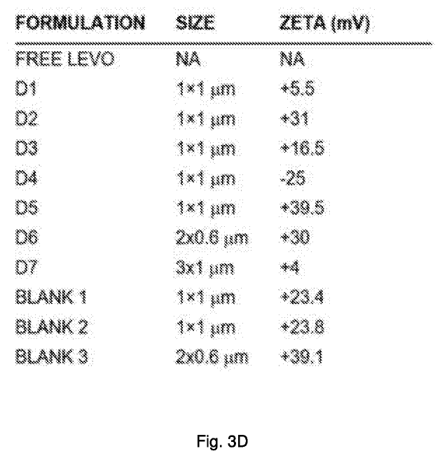

As shown in FIGS. 3A-D, all of the PRINT-Levofloxacin particles produced significant protection against E. coli infection in the tissue culture model. Even when washing of the cells after drug treatment was followed immediately by E. coli infection (FIG. 3A), the unencapsulated, free levofloxacin had limited efficacy in preventing E. coli intracellular infection of bladder epithelial cells. In contrast, PRINT-Levofloxacin abrogated the infection. At the test concentration (5 μg/ml), no difference in efficacy was observed regardless of formulation. The empty, blank PRINT particles had no inhibitory effect on the E. coli infection, indicating that the inhibition of infection by the antibiotic-containing formulations was attributable to the encapsulation of the antibiotic and not due to steric hindrance of bacterial adherence to the epithelial cells by the PRINT particles.

The inhibitory effect of the PRINT-Levofloxacin formulations was retained long after the initial single dose was applied to and removed from the tissue culture cells. After an 8 or 18 hour delay between washing the epithelial cells free of any unbound, uninternalized particles and the start of the infection (FIGS. 3B & 3C), the PRINT-Levofloxacin-treated bladder epithelial cells were largely protected against E. coli infection, with only 25% of the level of infection as media or free levofloxacin. These data demonstrate a strong depot-effect by the PRINT-Levofloxacin formulations that provide extended protection against intracellular infection.

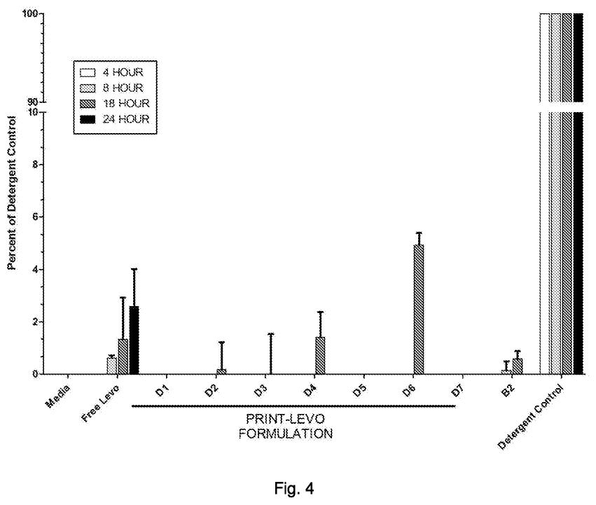

PRINT-Levofloxacin formulations are non-toxic to bladder epithelial cells. The prolonged efficacy of PRINT-Levofloxacin to protect bladder epithelial cells from infection was from a depot-effect where the cell-associated antibiotic particles slowly released levofloxacin over time. However, an alternative possibility was that the PRINT-Levofloxacin was toxic to the epithelial cells, making E. coli unable to persist within them and leading to the low observed bacterial counts after treatment. Cytotoxicity due to the PRINT-Levofloxacin formulations would impose significant limitations on their therapeutic use. Thus, lactate dehydrogenase (LDH) assays, measuring released cytosolic LDH in the supernatant of PRINT-Levofloxacin or control exposed cells as a measure of terminal cellular death was performed. As shown in FIG. 4, PRINT-Levofloxacin formulations were not more toxic than the same concentration of levofloxacin alone, even after 24 hours of exposure. Most of the formulations were less toxic than free levofloxacin over prolonged exposure, although the importance of the small amount of overall toxicity observed is not likely clinically significant. In total, these assays indicate that PRINT-Levofloxacin is non-toxic to bladder epithelial cells and that cytotoxicity does not account for the mechanism through which PRINT-Levofloxacin abrogates E. coli infection in cultured bladder epithelial cells.

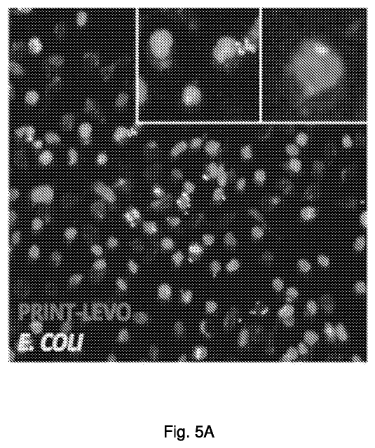

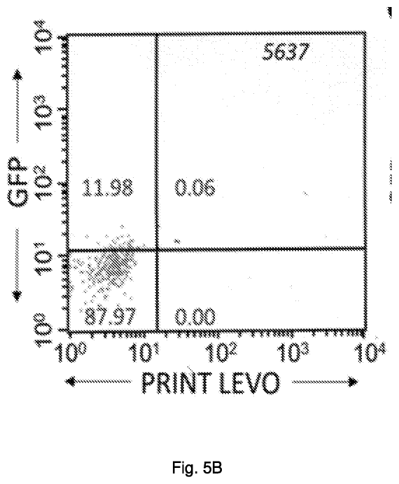

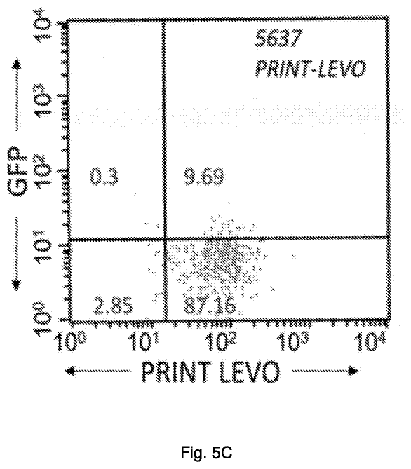

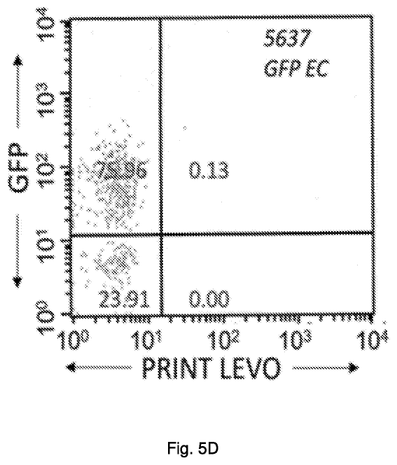

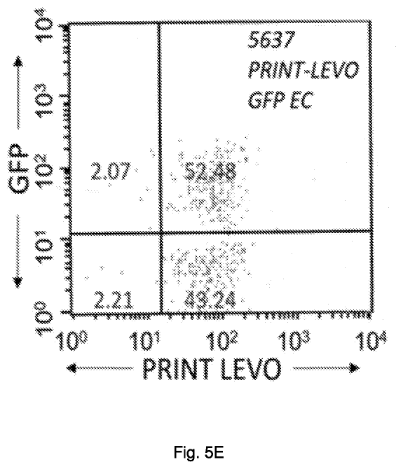

PRINT-Levofloxacin is found associated with the same cell as infecting E. coli but is not co-localized within the cell for its effectiveness. To test whether PRINT Levofloxacin was localized to the same cells as E. coli and co-localized with the bacteria within the epithelial cells, microscopy studies were performed by first incubating cultured bladder epithelium cell line 5637 with PRINT-Levofloxacin formula D8 containing a fluorescent Qdot for 1 hour. Non-cell associated D8 was removed with gentle washing followed by a 1 hour infection with GFP-expressing E. coli. Non-cell bound bacteria were washed away. The epithelial cells were fixed and examined by epifluorescence light microscopy. As shown in the representative image in FIG. 5A, PRINT-Levofloxacin was seen throughout the epithelium, frequently in small aggregates. However, as illustrated in the inset panels, D8 did not appear to be co-localized with the bacteria in most cases. These data may indicate that antibiotic unloaded from PRINT encapsulation is able to diffuse to the site of bacteria.

Based on the microscopy, it was unclear if all of the E. coli infected cells also had PRINT-LEVO or if drug from a PRINT-LEVO-carrying cell diffused into neighboring infected cells. We performed flow cytometry to distinguish the populations of D5-loaded and GFP-E. coli infected cells. We found that PRINT-Levofloxacin has inherent fluorescence detectable by flow cytometry and used this emission to track the particles. Bladder epithelial cell line 5637 was exposed to D5 and/or GFP-expressing E. coli in an identical fashion as used in the microscopy studies. The cells were fixed with paraformaldehyde at the conclusion of the experiment, and flow cytometry was performed. When bladder epithelial cells were incubated with D5, uniform labeling of majority of the cells was observed compared to untreated cells (FIGS. 5B vs. 5C). Similarly GFP-expressing E. coli infected a majority of the cells under the experimental conditions (FIG. 5D). When cells were first treated with D5 followed by infection with E. coli, it was observed that nearly all of the cells associated with GFP-producing E. coli cells were also labeled with D5. These data indicate that under our experimental conditions, D5 is broadly distributed among the bladder epithelial cells and is associated with most of the E. coli infected cells. Thus the D5 prophylactic effect is likely due to direct drug distribution to infected E. coli cells, resulting in the prevention of infection.

Single dose PRINT-levofloxcin provides prolonged protection against E. coli UTI in the SCI rat. Based on the efficacy of PRINT-Levofloxacin to provide extended protection to cultured bladder epithelial cells from E. coli infection, a comparison of the efficacy of PRINT-Levofloxacin formula D5 to free levofloxacin prophylaxis against UTI in the spinal cord-injured host was made. We employed the rat model of SCI superimposed with E. coli UTI, a model in which we previously demonstrated exquisite sensitivity of the animals to E. coli infection when compared to non-injured, normally voiding control animals. Thus this model reflects the human condition of neurogenic bladder [29].

PRINT-Levofloxacin D5 or free levofloxacin at a concentration of 20 μg/ml (based on total antibiotic) was administered to each animal by transurethral catheterization in a volume equal to the measured residual void volume for each animal, thus ensuring similar drug exposure throughout the bladder. Groups of control animals received PBS or blank PRINT particles of the same size, shape, and charge as D5. One hour after the administration of each test or control agent, urine was expelled from the rat bladders by crede to eliminate residual control or test agents. Four, eight, or eighteen hours after crede, the animals were infected with 104 kanamycin resistance-marked E. coli UT189 [29,30] by transurethral inoculation. Twenty four hours after the initiation of the infections, the animals were euthanized, and bladder and kidney homogenates were plated to enumerate the bacteria in each organ.

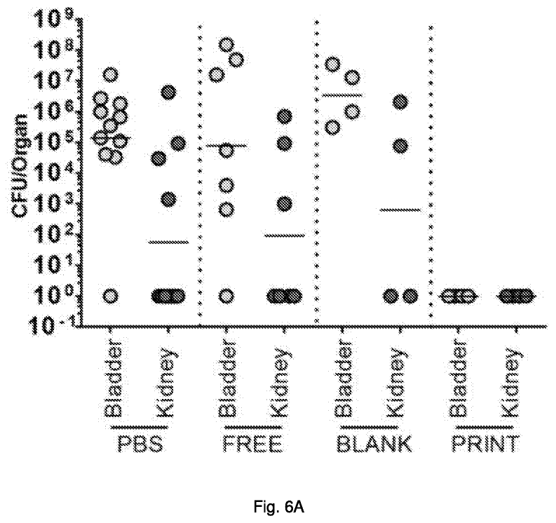

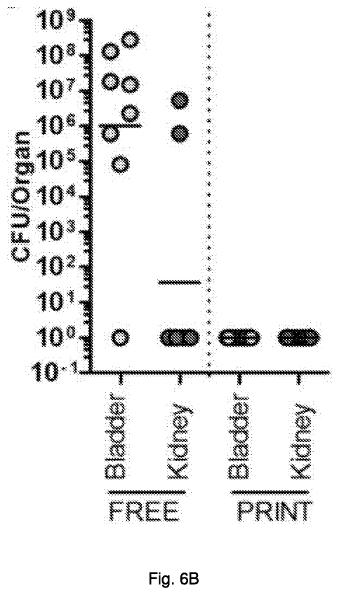

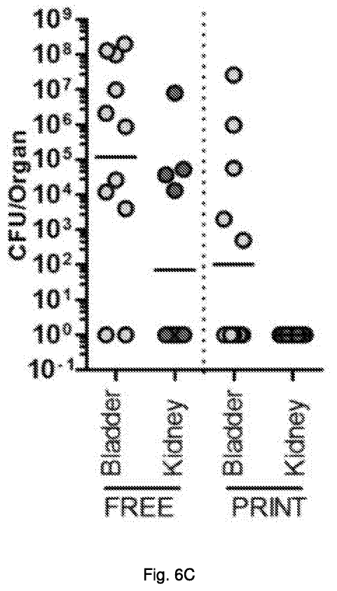

FIGS. 6A-C show the data for each time point. As shown in the 4 hour delay experiment, the PBS-treated control animals had high burdens of bacteria in almost all of the bladders (FIG. 6A). Consistent with our prior studies in the SCI rat, some but not all of the animals also had accompanying kidney infections. Neither BLANK PRINT particles nor free levofloxacin provided significant protection against infection in the 4 hour delay experiment. In contrast, the PRINT Levofloxacin treated animals were 100% infection-free in the bladders and kidneys (P<0.0001, FREE VS. PRINT). When the delay between emptying the therapeutic from the bladder and infection with E. coli was 8 hours, PRINT-Levofloxacin still provided 100% protection against E. coli infection compared to free levofloxacin-treated animals in which only 1 of 8 (12.5%) animals was infection-free (FIG. 6B; P<0.0001, FREE vs. PRINT for bladder and kidney). When the time between PRINT-Levofloxacin and E. coli infection was extended to 18 hours, the protective effect of the encapsulated antibiotic remained superior to free antibiotic (FIG. 6C; P<0.001 FREE vs. PRINT for bladder and kidney); however, the effect was diminished compared to shorter intervals between therapy and infection. The animals receiving PRINT-Levofloxacin had lower bladder counts compared to the animals receiving free antibiotic. The animals administered PRINT-Levofloxacin also had no infection of the kidneys, a notable difference in the infectious outcomes. These data indicated that a single administration of PRINT Levofloxacin can provide extended protection against upper and lower UTI in the SCI rat.

Efforts have been made to ensure accuracy with respect to numbers used (e.g. amounts, temperature, etc.) but some experimental errors and deviations should be accounted for.

One skilled in the art will recognize many methods and materials similar or equivalent to those described herein, which could be used in the practicing the subject matter described herein. The present disclosure is in no way limited to just the methods and materials described.

Unless defined otherwise, technical and scientific terms used herein have the same meaning as commonly understood by one of ordinary skill in the art to which this subject matter belongs, and are consistent with: Singleton et al (1994) Dictionary of Microbiology and Molecular Biology, 2nd Ed., J. Wiley & Sons, New York, N.Y.; and Janeway, C., Travers, P., Walport, M., Shlomchik (2001) Immunobiology, 5th Ed., Garland Publishing, New York.

Throughout this specification and the claims, the words “comprise,” “comprises,” and “comprising” are used in a non-exclusive sense, except where the context requires otherwise. It is understood that embodiments described herein include “consisting of” and/or “consisting essentially of” embodiments.

As used herein, the term “about,” when referring to a value is meant to encompass variations of, in some embodiments ±50%, in some embodiments+20%, in some embodiments+10%, in some embodiments+5%, in some embodiments+1%, in some embodiments ±0.5%, and in some embodiments ±0.1% from the specified amount, as such variations are appropriate to perform the disclosed methods or employ the disclosed compositions.

Where a range of values is provided, it is understood that each intervening value, to the tenth of the unit of the lower limit, unless the context clearly dictates otherwise, between the upper and lower limit of the range and any other stated or intervening value in that stated range, is encompassed. The upper and lower limits of these small ranges which may independently be included in the smaller rangers is also encompassed, subject to any specifically excluded limit in the stated range. Where the stated range includes one or both of the limits, ranges excluding either or both of those included limits are also included.

Many modifications and other embodiments set forth herein will come to mind to one skilled in the art to which this subject matter pertains having the benefit of the teachings presented in the foregoing descriptions and the associated drawings. Therefore, it is to be understood that the subject matter is not to be limited to the specific embodiments disclosed and that modifications and other embodiments are intended to be included within the scope of the appended claims. Although specific terms are employed herein, they are used in a generic and descriptive sense only and not for purposes of limitation.

### BIBLIOGRAPHY

1. Foxman B, Brown P (2003) Epidemiology of urinary tract infections: transmission and risk
   factors, incidence, and costs. Infect Dis Clin North Am 17: 227-241.
2. Foxman B (1990) Recurring urinary tract infection: incidence and risk factors. Am J Public

### Heal 80: 331-333.

3. Stamm W E, Hooton T M, Johnson J R, Johnson C, Stapleton A, et al. (1989) Urinary tract
   infections: from pathogenesis to treatment. J Infect Dis 159: 400-406.
4. McKibben M J, Seed P, Ross S S, Borawski K M (2015) Urinary Tract Infection and Neurogenic Bladder. Urol Clin North Am 42: 527-536. doi:10.1016/j.ucl.2015.05.006.
5. Siroky M B (2002) Pathogenesis of bacteriuria and infection in the spinal cord injured patient. Am J Med 113 Suppl: 67S-79S.
6. Cameron A P, Wallner L P, Tate D G, Sarma A V, Rodriguez G M, et al. (2010) Bladder management after spinal cord injury in the United States 1972 to 2005. J Urol 184: 213-217. doi:S0022-5347(10)02981-2 [pii]10.1016/j.juro.2010.03.008.
7. Mulvey M A, Schilling J D, Hultgren S J (2001) Establishment of a persistent Escherichia coli reservoir during the acute phase of a bladder infection. Infect Immun 69: 4572-4579.
8. Anderson G G, Palermo J J, Schilling J D, Roth R, Heuser J, et al. (2003) Intracellular bacterial biofilm-like pods in urinary tract infections. Science (80-) 301: 105-107.
9. Rosen D A, Hooton T M, Stamm W E, Humphrey P A, Hultgren S J (2007) Detection of intracellular bacterial communities in human urinary tract infection. PLoS Med 4: e329.
10. Rosen D A, Pinkner J S, Jones J M, Walker J N, Clegg S, et al. (2008) Utilization of an intracellular bacterial community pathway in Klebsiella pneumoniae urinary tract infection and the effects of FimK on type 1 pilus expression. Infect Immun 76: 3337-3345. doi:IAI.00090-08 [pii]10.1128/IAI.00090-08.
11. Darouiche R O, Thornby J I, Cerra-Stewart C, Donovan W H, Hull R A (2005) Bacterial interference for prevention of urinary tract infection: a prospective, randomized, placebo controlled, double-blind pilot trial. Clin Infect Dis 41: 1531-1534. doi:10.1086/497272.
12. Darouiche R O, Green B G, Donovan W H, Chen D, Schwartz M, et al. (2011) Multicenter
   randomized controlled trial of bacterial interference for prevention of urinary tract infection in patients with neurogenic bladder. Urology 78: 341-346. doi:10.1016/j.urology.2011.03.062.
13. Warren J W, Platt R, Thomas R J, Rosner B, Kass E H (1978) Antibiotic irrigation and catheter-associated urinary-tract infections. N Engl J Med 299: 570-573. doi:10.1056/NEJM197809142991103.
14. Defoor W, Ferguson D, Mashni S, Creelman L, Reeves D, et al. (2006) Safety of gentamicin bladder irrigations in complex urological cases. J Urol 175: 1861-1864. doi:10.1016/S0022-5347(05)00928-6.
15. Huynh D, Morgan J A (2011) Use of intravesicular amikacin irrigations for the treatment
   and prophylaxis of urinary tract infections in a patient with spina bifida and neurogenic bladder: a case report. J Pediatr Pharmacol Ther 16: 102-107. doi:10.5863/1551-6776-16.2.102.
16. Zdziarski J, Brzuszkiewicz E, Wullt B, Liesegang H, Biran D, et al. (2010) Host imprints
   on bacterial genomes—rapid, divergent evolution in individual patients. PLoS Pathog 6: e1001078. doi:10.1371/journal.ppat.1001078.
17. Sundén F, Håkansson L, Ljunggren E, Wullt B (2006) Bacterial interference—is deliberate
   colonization with Escherichia coli 83972 an alternative treatment for patients with recurrent urinary tract infection? Int J Antimicrob Agents 28 Suppl 1: S26-S29. doi:10.1016/j.ijantimicag.2006.05.007.
18. Johnson L, Sabel A, Burman W J, Everhart R M, Rome M, et al. (2008) Emergence of fluoroquinolone resistance in outpatient urinary Escherichia coli isolates. Am J Med 121: 876-884.
19. McCusker M E, Harris A D, Perencevich E, Roghmann M-C (2003) Fluoroquinolone use
   and Clostridium difficile-associated diarrhea. Emerg Infect Dis 9: 730-733. doi:10.3201/eid0906.020385.
20. Euliss L E, DuPont J A, Gratton S, DeSimone J (2006) Imparting size, shape, and composition control of materials for nanomedicine. Chem Soc Rev 35: 1095-1104. doi:10.1039/b600913c.
21. Rolland J P, Van Dam R M, Schorzman D A, Quake S R, DeSimone J M (2004) Solvent resistant photocurable liquid fluoropolymers for microfluidic device fabrication [corrected]. J Am Chem Soc 126: 2322-2323. doi:10.1021/ja031657y.
22. Rolland J P, Maynor B W, Euliss L E, Exner A E, Denison G M, et al. (2005) Direct fabrication and harvesting of monodisperse, shape-specific nanobiomaterials. J Am Chem Soc 127: 10096-10100. doi:10.1021/ja051977c.
23. Wang J, Tian S, Petros R A, Napier M E, Desimone J M (2010) The complex role of multivalency in nanoparticles targeting the transferrin receptor for cancer therapies. J Am Chem Soc 132: 11306-11313. doi:10.1021/ja1043177.
24. Mulvey M A, Lopez-Boado Y S, Wilson C L, Roth R, Parks W C, et al. (1998) Induction and
   evasion of host defenses by type 1-piliated uropathogenic Escherichia coli. Science (80-) 282: 1494-1497.
25. Anderson G G, Goller C C, Justice S, Hultgren S J, Seed P C (2010) Polysaccharide Capsule and Sialic Acid-Mediated Regulation Promote Biofilm-like Intracellular Bacterial Communities During Cystitis. Infect Immun 78: 963-975. doi:IAI.00925-09 [pii] 10.1128/IAI.00925-09.
26. Schwartz D J, Chen S L, Hultgren S J, Seed P C (2011) Population dynamics and niche distribution of uropathogenic Escherichia coli during acute and chronic urinary tract infection. Infect Immun 79: 4250-4259. doi:10.1128/IAI.05339-11.
27. Mills M, Meysick K C, O'Brien A D (2000) Cytotoxic necrotizing factor type 1 of uropathogenic Escherichia coli kills cultured human uroepithelial 5637 cells by an apoptotic mechanism. Infect Immun 68: 5869-5880.
28. Petzoldt J L, Leigh I M, Duffy P G, Sexton C, Masters J R (1995) Immortalisation of human
   urothelial cells. Urol Res 23: 377-380.
29. Balsara Z R, Ross S S, Dolber P C, Wiener J S, Tang Y, et al. (2013) Enhanced susceptibility to urinary tract infection in the spinal cord-injured host with neurogenic bladder. Infect Immun 81: 3018-3026. doi:10.1128/IAI.00255-13.
30. Chaudhry R, Madden-Fuentes R J, Ortiz T K, Balsara Z, Tang Y, et al. (2013) Inflammatory Response to Escherichia coli Urinary Tract Infection in the Neurogenic Bladder of the Spinal Cord Injured Host. J Urol. doi:10.1016/j.juro.2013.12.013.
31. Schilling J D, Mulvey M A, Vincent C D, Lorenz R G, Hultgren S J (2001) Bacterial invasion
   augments epithelial cytokine responses to Escherichia coli through a lipopolysaccharide dependent mechanism. J Immunol 166: 1148-1155.
32. Schilling J D, Martin S M, Hunstad D A, Patel K P, Mulvey M A, et al. (2003) CD14- and Toll
   like receptor-dependent activation of bladder epithelial cells by lipopolysaccharide and type 1 piliated Escherichia coli. Infect Immun 71: 1470-1480.
33. Martinez J J, Hultgren S J (2002) Requirement of Rho-family GTPases in the invasion of
   Type 1-piliated uropathogenic Escherichia coli. Cell Microbiol 4: 19-28. doi:166 [pii].
34. Schilling J D, Mulvey M A, Hultgren S J (2001) Structure and function of Escherichia coli
   type 1 pili: new insight into the pathogenesis of urinary tract infections. J Infect Dis 183: S36-S40.
35. Martinez J J, Mulvey M A, Schilling J D, Pinkner J S, Hultgren S J (2000) Type 1 pilus mediated bacterial invasion of bladder epithelial cells. EMBO J 19: 2803-2812.

1.-20. (canceled)

21. A method of preventing or reducing severity of a urinary tract infection in a subject in need thereof, the method comprising:
administering a therapeutically effective amount of a plurality of particles, wherein each particle of the plurality comprises:
a biocompatible matrix comprising:
poly(D,L-lactide-co-glycolide),
a cationic agent and/or a pharmaceutically acceptable salt thereof; and
an antibiotic and/or pharmaceutically acceptable salt thereof, wherein the antibiotic is dispersed substantially throughout the biocompatible matrix; and
a non-spherical three-dimensional engineered shape comprising:
at least two substantially planar surfaces, and
in cross-section, a maximum dimension of less than about 10 μm;
wherein administration provides protection to the urinary bladder from 104 kanamycin resistance-marked E. coli UTI189 for at least eight hours.

22. The method of claim 21, wherein the administration provides protection to the kidneys from 104 kanamycin resistance-marked E. coli UTI189 for at least eighteen hours.

23. The method of claim 21, wherein administration comprises instilling the plurality of particles into the bladder of a subject and expelling urine from the subject approximately one hour after instillation.

24. The method of claim 21, wherein the poly(D,L-lactide-co-glycolide) comprises a molecular weight average (M_{w}) of about 16 kDa to about 54 kDa.

25. The method of claim 21, wherein the poly(D,L-lactide-co-glycolide) comprises a molecular weight average (M_{w}) of about 24 kDa to about 38 kDa.

26. The method of claim 21, wherein the poly(D,L-lactide-co-glycolide) comprises a molecular weight average (M_{w}) of about 6 kDa.

27. The method of claim 21, wherein the poly(D,L-lactide-co-glycolide) comprises a molar ratio of D,L-lactide:glycolide of about 50:50.

28. The method of claim 21, wherein the cationic agent and/or pharmaceutically acceptable salt thereof is selected from the group consisting of cationic lipids, cationic polymers, cationic lipidoids, and cationic agents containing a portion having a positive charge in aqueous solutions at neutral pH.

29. The method of claim 21, wherein the antibiotic and/or pharmaceutically acceptable salt thereof comprises a quinolone or a fluoroquinolone.

30. The method of claim 21, wherein the antibiotic and/or pharmaceutically acceptable salt thereof comprises levofloxacin.

31. The method of claim 21, wherein the antibiotic and/or pharmaceutically acceptable salt thereof comprises levofloxacin and the levofloxacin comprises up to about 13.5 wt % of the particle.

32. The method of claim 21, wherein the non-spherical three-dimensional engineered shape comprises a rod.

33. The method of claim 32, wherein the rod size is selected from approximately d=1 μm×h=1 μm, d=2 μm×h=0.6 μm, and d=3 μm×h=1 μm; wherein d represents the diameter and h represents the height of the rod.

34. The method of claim 21, wherein administration is via infiltration.

35. The method of claim 21, wherein administration provides topical, intracystic prophylaxis against urinary tract infection for subjects having a neurogenic bladder.

36. A method of preventing or reducing severity of a urinary tract infection in a subject in need thereof, the method comprising:
administering a therapeutically effective amount of a plurality of particles, wherein each particle of the plurality comprises:
a biocompatible matrix comprising:
poly(D,L-lactide-co-glycolide) comprising a molecular weight average (Mw) of about 16 kDa to about 54 kDa,
1,2-dioleoyl-3-trimethylammonium-propane (DOTAP) and/or a pharmaceutically acceptable salt thereof, wherein a pre-particle solution used to fabricate the particle comprises about 0 wt % to about 5 wt % DOTAP; and
levofloxacin and/or pharmaceutically acceptable salt thereof, wherein:
levofloxacin comprises about 3.5 wt % to about 13.5 wt % of the particle and
the antibiotic is dispersed substantially throughout the biocompatible matrix; and
a non-spherical three-dimensional engineered shape comprising:
at least two substantially planar surfaces, and
in cross-section, a maximum dimension of less than about 10 μm; and
wherein administration provides protection to the urinary bladder from 104 kanamycin resistance-marked E. coli UTI189 for at least eight hours.

37. A method of preventing or reducing severity of a urinary tract infection in a subject in need thereof, the method comprising:
administering a therapeutically effective amount of a plurality of particles, wherein each particle of the plurality comprises:
a biocompatible matrix comprising:
a biocompatible polymer,
a cationic agent and/or a pharmaceutically acceptable salt thereof;
and
an antibiotic and/or pharmaceutically acceptable salt thereof, wherein the antibiotic is dispersed substantially throughout the biocompatible matrix; and
a non-spherical three-dimensional engineered shape comprising:
at least two substantially planar surfaces, and
in cross-section, a maximum dimension of less than about 10 μm;
wherein administration reduces severity of urinary bladder infection up to 58 percent at eighteen hours post-administration.

38. The method of claim 37, wherein administration comprises instilling the plurality of particles into the bladder of a subject.

39. The method of claim 37, wherein the antibiotic and/or pharmaceutically acceptable salt thereof comprises a quinolone or a fluoroquinolone.

40. The method of claim 37, wherein the antibiotic and/or pharmaceutically acceptable salt thereof comprises up to about 13.5 wt % of the particle.
